(19) United States
(12) Patent Application Publication
(10) Pub. No.: US 2020/0261534 A1
(43) Pub. Date: Aug. 20, 2020

(54) NEW TREATMENTS OF MULTIPLE MYELOMA

(71) Applicant: IDP DISCOVERY PHARMA, S.L., Barcelona (ES)

(72) Inventors: Santiago ESTEBAN MARTÍN, Barcelona (ES); Laura NEVOLA, Barcelona (ES); Enrique María OCIO SAN MIGUEL, Salamanca (ES); Patryk KRZEMINSKI, Salamanca (ES); Mercedes GARAYOA, Salamanca (ES)

(21) Appl. No.: 16/651,983

(22) PCT Filed: Sep. 10, 2018
(86) PCT No.: PCT/EP2018/074317

§ 371 Date: Mar. 27, 2020

(30) Foreign Application Priority Data
Sep. 8, 2017 (EP) 17382601.7

(51) Int. Cl. A61K 38/10; A61K 31/573; A61K 31/69; A61K 31/675; A61P 35/00

(52) U.S. Cl. CPC A61K 38/10; A61K 31/573; A61P 35/00; A61K 31/675; A61K 31/69

(57) ABSTRACT

The present invention relates to the field of oncology. More specifically, it relates to the use of peptidomimetic compounds in the treatment of multiple myeloma, especially in refractory or relapsing multiple myeloma and/or in combination with other antitumoral agents.

### FIELD OF INVENTION

The present invention relates to the field of oncology. More specifically, it relates to the use of peptidomimetic compounds in the treatment of multiple myeloma, especially in resistant, refractory or relapsing multiple myeloma and/or in combination with other antitumoral agents.

### BACKGROUND OF THE INVENTION

Multiple myeloma is a malignant neoplasm of plasma cells that accumulate in bone marrow, leading to bone destruction and marrow failure. Like plasma cells, myeloma cells also make antibodies, but they are all copies of one specific type of antibody. Since these are made from a single clone, they are called monoclonal proteins or M-proteins.

Myeloma cells tend to collect in the bone marrow and in the hard outer part of bones. A mass of myeloma cells is called a plasmacytoma. When there is only one mass of myeloma cells, it is generally referred as a solitary plasmacytoma. In most cases, however, the myeloma cells grow and spread throughout the bone marrow, invade bone tissue, and spread all over the body. When this happens, the disease is called multiple myeloma. The terms multiple myeloma and myeloma are used herein interchangeably.

Multiple myeloma may cause a number of organ dysfunctions and symptoms including bone pain or fracture, renal failure, susceptibility to infection, anemia, hypercalcemia, and occasionally clotting abnormalities, neurologic symptoms, and vascular manifestations of hyperviscosity. Multiple myeloma is the second most commonly diagnosed hematologic malignancy in the Western World, with an incidence of 30,000 new cases in the U.S. alone in 2016 according to the National Cancer Institute (https://seer.cancer.gov/statfacts/html/mulmy.html). Unfortunately, multiple myeloma is presently considered an incurable disease and the overall survival of patients has remained essentially unchanged at a median of 3-4 years, despite intense efforts over the last 3 decades to improve on the activity of cytotoxic chemotherapy-based therapies for this disease.

Recently, there have been a series of important advances in the therapeutic management of multiple myeloma with the introduction as treatment options of thalidomide, and its immunomodulatory derivatives such as lenalidomide (Dimopoulos M et al. N. Engl. J. Med. 2007, 357, 2123-2132; Weber D M et al. N. Engl. J. Med. 2007, 357, 2133-2142) and proteasome inhibitors such as bortezomib (Richardson P G et al. N. Engl. J. Med. 2005, 352, 2487-2498). Although these classes of agents have been shown to be active in the setting of multiple myeloma patients who relapsed or were refractory to conventional or high-dose cytotoxic chemotherapy-based regimens, a significant proportion of multiple myeloma patients has de novo resistance to these agents, while initial responders (even those achieving durable complete remissions) can eventually relapse. Therefore the development of novel classes of agents against multiple myeloma is urgently needed, in order to further improve the outcome of multiple myeloma patients and, hopefully, to achieve high cure rates for this presently incurable neoplasia.

More information about multiple myeloma can be found in the medical literature such as in the Devita, Hellman, and Rosenberg's cancer: principles & practice of oncology, 10 edition, December 2014, Wolters Kluwer; the NCCN guidelines insights multiple myeloma, version 3.2016 (J Natl Compr Canc Netw 2016; 14(4):389-400), and Multiple Myeloma, version 3.2017 (J Natl Compr Canc Netw 2017; 15:230-269).

The ideal antitumor drug would kill cancer cells selectively, with a wide index relative to its toxicity towards non-cancer cells, and would also retain its efficacy against cancer cells, even after prolonged exposure to the drug. Unfortunately, none of the current therapies with known agents has an ideal profile. Most chemotherapeutic agents have very narrow therapeutic indexes and, in addition, cancerous cells exposed to slightly sublethal concentrations may develop resistance to such an agent, and quite often cross-resistance to several other antitumor agents.

Anticancer peptides and peptidomimetics (or peptide-like molecules) have become promising molecules as novel anticancer agents. A peptidomimetic is a compound containing non-peptidic structural elements (e.g., unnatural amino acids) that is designed to mimic or to antagonize the biological activity of a natural peptide. Peptidomimetics can be obtained from structural modifications of the parent peptide in order to alter its properties. As a consequence of these modifications, peptidomimetics no longer have classical peptide characteristics such as enzymatically labile peptidic bonds, and for this reason one of the main applications of peptidomimetic design is to increase the stability of the parent peptide.

c-Myc is a nuclear protein with important roles in cell growth and differentiation and its aberrant overexpression has been associated to carcinogenesis (Meichle A., Biochim. Biophys Acta, 1114: 129-146, 1992). More specifically, c-Myc has been described to be a transcription factor that heterodimerizes with Max to transactivate target downstream effectors (Blackwood E M, et al., Curr Opin Genet Dev. 1992, 2(2):227-35; Cole M D and Mc Mahon S B, Oncogene. 1999 May 13; 18(19):2916-24).

Huang et al. (Exp Hematol 2006, 34(11): 1480-9) have reported the small molecule compound 10058-F4 as an inhibitor of c-Myc/Max dimerization which showed anti-proliferative properties in vitro in acute myeloid leukemia cells. Peptidomimetic compounds have been previously described in WO2010/034031, which discloses Myc peptidomimetic macrocycles designed for binding to Max and its potential use in the treatment of proliferative diseases. This document does not show however that these peptides actually inhibit c-Myc/Max transactivating activity, let alone that these peptidomimetic agents have tumor inhibiting properties.

Despite new treatment options having become available in the recent years, there is still a need to find new drugs and drug combinations with reduced side-effects and improved efficacy in the treatment of newly diagnosed multiple myeloma and in particular for treating refractory and relapsing multiple myeloma.

### SUMMARY OF THE INVENTION

The present invention provides peptidomimetic compounds for use in the treatment of multiple myeloma.

Example 1 shows the anti-tumoral activity in vitro of a compound of formula I (e.g. S09 and S014) in various cancer cell lines, including the multiple myeloma cell line MM.S1. Moreover, in Example 2 is shown that the cross-linking with a ligand in positions X1 and X3 of a peptide of formula (I) is essential to the observed cytotoxic activity, a loss of efficacy being observed when the ligand is attached to other positions of the peptide sequence.

In addition, a compound of formula I has shown in Example 3 to have cytotoxic effects in the uM range in several multiple myeloma cell lines, including alkylating agent, corticosteroid and anthracycline resistant cell lines. Most patients with multiple myeloma experience relapse or are refractory to treatment. The ability of a compound of formula I to inhibit tumour growth in cell lines resistant to drugs generally used in treatment regimens for multiple myeloma suggests its usefulness in the treatment of such patients, for instance as second, third or further line treatment, as single agent or in combination with other drugs.

Moreover, in Example 4, S09 showed very good in vitro synergy with standard of care anti-myeloma agents. In particular, the triple combination treatments with S09, dexamethasone, and a drug selected from either bortezomib or cyclophosphamide, presented synergism as indicated by the calculated combination index (CI) which was substantially below 1 at each of the S09 concentrations tested.

Also, in Example 5, S09 was shown to potentiate in vivo the antiproliferative effects of bortezomib, dexamethasone and cyclophosphamide in double and triple combinations.

Accordingly, the first aspect of the invention relates to a compound of formula (I):

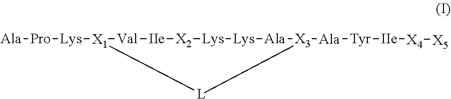

wherein X₂ is a non-polar amino acid, preferably selected from the group consisting of Leu and Phe; and

wherein X₄ is an amino acid, preferably Leu;

wherein X₅ is an amino acid, preferably Ser;

wherein X₁ and X₃ are independently selected and have formula (II):

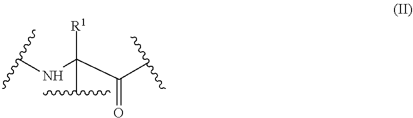

wherein R₁ is H or a monoradical selected from the group consisting of: (C₁-C₁₀)alkyl, (C₂-C₁₀)alkenyl, (C₂-C₁₀)alkynyl, (C₁-C₁₀)alkyl-O—(C₁-C₁₀)alkyl, (C₁-C₁₀)alkyl-C(═O)—(C₁-C₁₀)alkyl, (C₁-C₁₀)alkyl-O—C(O)—(C₁-C₁₀)alkyl, (C₁-C₁₀)alkyl-C(O)—NR₂—(C₁-C₁₀)alkyl, (C₁-C₁₀)alkyl-S—(C₁-C₁₀)alkyl, (C₁-C₁₀)alkyl-SR₃—(C₁-C₁₀)alkyl, (C₁-C₁₀)alkyl-S(═O)₂—(C₁-C₁₀)alkyl, (C₁-C₁₀)alkyl-S(═O)—(C₁-C₁₀)alkyl, (C₁-C₁₀)alkyl-O—S(═O)₂—O—(C₁-C₁₀)alkyl, (C₁-C₁₀)alkyl-NR₄—(C₁-C₁₀)alkyl; and a (preferably known) ring system comprising from 3 to 14 carbon atoms, the system comprising from 1 to 3 rings, where:
   each one of the rings is saturated, partially unsaturated, or aromatic;
   the rings are isolated, partially or totally fused,
   each one of the members forming the (preferably known) ring system is selected from the group consisting of: —CH—, —CH₂—, —NH—, —N—, —SH—, —S—, and —O—; and
   the ring system is optionally substituted by one or more radicals independently selected from the group consisting of halogen, —OH, —NO₂, (C₁-C₁₀)alkyl, (C₁-C₁₀)haloalkyl, and (C₁-C₁₀)alkyl-O—; and
R₂, R₃ and R₄ are monoradicals independently selected from the group consisting of: hydrogen, (C₁-C₁₀)alkyl, (C₂-C₁₀)alkenyl, and (C₂-C₁₀)alkynyl; and
wherein L is a biradical bound to X₁ and X₃ via the alpha carbon, and is selected from the group consisting of: —O—, O—(C₁-C₁₀)alkyl-O—, O—(C₁-C₁₀)alkenyl-O—, C(═O), C(═O)NR₅, C(═O)O, NR₆, S—S—, S—(C₁-C₁₀)alkyl-S, S—(C₁-C₁₀)alkenyl-S— and a (preferably known) ring system consisting of one ring from 3 to 6 members, the ring:
   being saturated, partially unsaturated, or aromatic;
   each one of the members forming the (preferably known) ring system being selected from the group consisting of: —CH—, —CH₂—, —NH—, —N—, —SH—, —S—, and —O—; and
   the ring system being optionally substituted by one or more radicals independently selected from the group consisting of halogen, —OH, —NO₂, (C₁-C₁₀)alkyl, (C₁-C₁₀)haloalkyl, and (C₁-C₁₀)alkyl-O—; and
R₅ and R₆ are radicals independently selected from the group consisting of: —H and (C₁-C₁₀)alkyl (C₁-C₁₀)alkyl, (C₂-C₁₀)alkenyl, and (C₂-C₁₀)alkynyl;

for use in a method of treating a subject having multiple myeloma.

It also relates to the use of a compound of formula (I) in the manufacturing of a medicament for the treatment of multiple myeloma.

In addition, the present invention provides a method of treating multiple myeloma comprising administering to a subject in need of such treatment a therapeutically effective amount of a compound of formula (I).

The invention also provides a pharmaceutical composition comprising a compound of formula (I), and a pharmaceutically acceptable carrier or excipient, for use in a method of treating a subject having multiple myeloma.

In a particular embodiment of any of the above, said multiple myeloma is resistant, refractory or relapsing multiple myeloma, preferably wherein said multiple myeloma is resistant, refractory or relapsing to a previous treatment (e.g. chemotherapy, targeted therapy or corticosteroids).

In another particular embodiment, optionally in combination with one or more of the embodiments or features mentioned above or below, said compound of formula (I) is administered in combination with another drug.

The invention is also directed to the use of a compound of formula (I) for the manufacture of a medicament for an effective treatment of cancer by combination therapy employing a compound of formula (I) with another drug.

It is further directed to a method of treating cancer comprising administering to a patient in need of such treatment a therapeutically effective amount of a compound of formula (I), in combination with a therapeutically effective amount of another drug.

The invention also provides a pharmaceutical composition comprising a compound of formula (I), another drug, and a pharmaceutically acceptable carrier or excipient, for use in combination in a method of treating multiple myeloma as described herein.

The invention further provides a kit for use in a method of treating multiple myeloma as described herein, which comprises: a pharmaceutical composition comprising a compound of formula (I), and a pharmaceutical composition comprising another drug; and optionally, instructions for use of both drugs in combination in a method of treating multiple myeloma.

In a particular embodiment of any of the above, said other drug is an anticancer drug, preferably is selected from the group consisting of an alkylating agent, a corticosteroid and a proteasome inhibitor, and combinations thereof.

Preferably, the present invention is concerned with synergistic combinations of a compound, pharmaceutical composition or kit as described herein.

### BRIEF DESCRIPTION OF THE DRAWINGS

FIG. 1: Double and triple combinations of S09, dexamethasone (DEXA) 5 nM and bortezomib (BORT) 1 nM.

This figure illustrates the percentage of cell survival of the MM1S cell line obtained further to 24 h incubation with S09 (at 0 uM, 0.3 uM, 0.6 uM, 1.25 uM and 2.5 uM) as single agent, in double combinations with DEXA 5 nM and BORT nM, respectively, and in the triple combination.

FIG. 2: Double and triple combinations of S09, dexamethasone (DEXA) 5 nM and bortezomib (BORT) 2 nM.

This figure illustrates the percentage of cell survival of the MM1S cell line obtained further to 24 h incubation with S09 (at 0 uM, 0.3 uM, 0.6 uM, 1.25 uM and 2.5 uM) as single agent, in double combinations with DEXA 5 nM and BORT 2 nM, respectively, and in the triple combination.

FIG. 3: Double and triple combinations of S09, dexamethasone (DEXA) 5 nM and cyclophosphamide (CYCLO) 2.5 uM.

This figure illustrates the percentage of cell survival of the MM1S cell line obtained further to 24 h incubation with S09 (at 0 uM, 0.3 uM, 0.6 uM, 1.25 uM and 2.5 uM) as single agent, in double combinations with DEXA 5 nM and CYCLO 2.5 uM, respectively, and in the triple combination.

FIG. 4: Double and triple combinations of S09, dexamethasone (DEXA) 5 nM and cyclophosphamide (CYCLO) 5 uM.

This figure illustrates the percentage of cell survival of the MM1S cell line obtained further to 24 h incubation with S09 (at 0 uM, 0.3 uM, 0.6 uM, 1.25 uM and 2.5 uM) as single agent, in double combinations with DEXA 5 nM and CYCLO 5 uM, respectively, and in the triple combination.

### DETAILED DESCRIPTION OF THE INVENTION

### Definitions

The term “amino acid” refers to a molecule containing both an amino group and a carboxyl group. Unless otherwise explicitly stated, the amino acid can have L- or D-configuration. Amino acids can be classified by the side chain group. There are basically four different classes of amino acids determined by different side chains: (1) non-polar, (2) polar and neutral (uncharged polar), (3) acidic and polar (hereinafter also referred as “acid” or “acidic” amino acids), (4) basic and polar (hereinafter also referred as “basic” amino acids).

Non-polar amino acids have side chains which are hydrocarbon alkyl groups (alkane branches) or aromatic (benzene rings) or heteroaromatic (e.g. indole ring). Illustrative non-limitative examples of common non-polar amino acids are Ala, Val, Leu, lie, Pro, Trp, Gly, Phe, and Met.

Polar-neutral amino acids have polar but not charged groups at neutral pH in the side chain (such as hydroxyl, amide or thiol groups). Illustrative non-limitative examples of polar neutral amino acids are Ser, Thr, Cys, Tyr, Asn, and Gin.

In certain embodiments, an amino acid is an alpha amino acid. Suitable amino acids include, without limitation, natural alpha-amino acids such as L-isomers of the 20 common naturally occurring alpha-amino acids: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine; natural beta-amino acids (e.g., beta-alanine); and unnatural amino acids.

TABLE 1

| Exemplary | Suitable amino acid | |
|---|---|---|
| unnatural | side chains | |
| alpha-amino acids | R | R′ |
| D-Alanine | —H | —CH₂ |
| D-Arginine | —H | —CH₂CH₂CH₂—NHC(═NH)NH₂ |
| D-Asparagine | —H | —CH₂C(═O)NH₂ |
| D-Aspartic acid | —H | —CH₂CO₂H |
| D-Cysteine | —H | —CH₂SH |
| D-Glutamic acid | —H | —CH₂CH₂CO₂H |
| D-Glutamine | —H | —CH₂CH₂C(═O)NH₂ |
| D-Histidine | —H | —CH₂-2-(1H-imidazole) |
| D-Isoleucine | —H | -sec-butyl |
| D-Leucine | —H | -iso-butyl |
| D-Lysine | —H | —CH₂CH₂CH₂CH₂NH₂ |
| D-Methionine | —H | —CH₂CH₂SCH₃ |
| D-Phenylalanine | —H | —CH₃Ph |
| D-Proline | —H | -2-(pyrrolidine) |
| D-Serine | —H | —CH₂OH |
| D-Threonine | —H | —CH₂CH(OH)(CH₃) |
| D-Tryptophan | —H | —CH₂-3-(1H-indole) |
| D-Tyrosine | —H | —CH₂-(p-hydroxyphenyl) |
| D-Valine | —H | -isopropyl |
| Di-vinyl | —CH═CH₂ | —CH═CH₂ |
| α-methyl-Alanine | —CH₃ | —CH₃ |
| (Aib) | | |
| α-methyl-Arginine | —CH₃ | —CH₂CH₂CH₂—NHC(═NH)NH₂ |
| α-methyl-Asparagine | —CH₃ | —CH₂C(═O)NH₂ |
| α-methyl-Aspartic | —CH₃ | —CH₂CO₂H |
| acid | | |
| α-methyl-Cysteine | —CH₃ | —CH₂SH |
| α-methyl-Glutamic | —CH₃ | —CH₂CH₂CO₂H |
| acid | | |
| α-methyl-Glutamine | —CH₃ | —CH₂CH₂C(═O)NH₂ |
| α-methyl-Histidine | —CH₃ | —CH₂-2-(1H-imidazole) |
| α-methyl-Isoleucine | —CH₃ | -sec-butyl |
| α-methyl-Leucine | —CH₃ | -iso-butyl |
| α-methyl-Lysine | —CH₃ | —CH₂CH₂CH₂CH₂NH₂ |

TABLE 2
Unnatural amino acids

| | |
|---|---|
| Aad | 2-Aminoadipic acid |
| bAad | 3-Aminoadipic acid |
| bAla | beta-Alanine, beta-Aminopropionic acid |
| Abu | 2-Aminobutyric acid |
| 4Abu | 4-Aminobutyric acid, piperidinic acid |
| Acp | 6-Aminocaproic acid |
| Ahe | 2-Aminoheptanoic acid |
| Aib | 2-Aminoisobutyric acid |
| bAib | 3-Aminoisobutyric acid |
| Apm | 2-Aminopimelic acid |
| Dbu | 2,4 Diaminobutyric acid |
| Des | Desmosine |
| Dpm | 2,2′-Diaminopimelic acid |
| Dpr | 2,3-Diaminopropionic acid |
| EtGly | N-Ethylglycine |
| EtAsn | N-Ethylasparagine |
| Hvl | Hydroxylysine |
| aHyl | allo-Hydroxylysine |
| 3Hyp | 3-Hydroxyproline |
| 4Hyp | 4-Hydroxyproline |
| Ide | Isodesmosine |
| alle | allo-Isoleucine |
| MeGly | N-Methylglycine, sarcosine |
| MeIle | N-Methylisoleucine |
| MeLvs | 6-N-Methyllysine |
| MteVal | N-Methylvaline |
| Nva | Norvaline |
| Nle | Norleucine |
| Orn | Ornithine |

Amino acids used in the construction of peptides of the present invention may be prepared by organic synthesis, or obtained by other routes, such as, for example, degradation of or isolation from a natural source.

There are many known unnatural amino acids any of which may be included in the peptides of the present invention (some of them are listed in Table 2 above). Some examples of unnatural amino acids are 4-hydroxyproline, desmosine, gamma-aminobutyric acid, beta-cyanoalanine, norvaline, 4-(E)-butenyl-4(R)-methyl-N-methyl-L-threonine, N-methyl-L-leucine, 1-amino-cyclopropanecarboxylic acid, 1-amino-2-phenyl-cyclopropanecarboxylic acid, 1-amino-cyclobutanecarboxylic acid, 4-amino-cyclopentenecarboxylic acid, 3-amino-cyclohexanecarboxylic acid, 4-piperidylacetic acid, 4-amino-1-methylpyrrole-2-carboxylic acid, 2,4-diaminobutyric acid, 2,3-diaminopropionic acid, 2,4-diaminobutyric acid, 2-aminoheptanedioic acid, 4-(aminomethyl)benzoic acid, 4-aminobenzoic acid, ortho-, meta- and para-substituted phenylalanines (e.g., substituted with —C(═O)C₆H₅; —CF₃; —CN; -halo; —NO₂; —CH₃), disubstituted phenylalanines, substituted tyro sines (e.g., further substituted with —C(═O)C₆H₅; —CF₃; —CN; -halo; —NO₂; —CH₃), and statine. Additionally, the amino acids suitable for use in the present invention may be derivatized to include amino acid residues that are chemically modified, such as hydroxylated, phosphorylated, sulfonated, acylated, lipidated, and glycosylated amino acid residues.

The term “treating”, as used herein, unless otherwise indicated, includes the amelioration, cure, and/or maintenance of a cure (i.e., the prevention or delay of relapse) of a disease or disorder. Treatment after a disorder has started aims to reduce, alleviate, ameliorate or altogether eliminate the disorder, and/or its associated symptoms, to prevent it from becoming worse, to slow the rate of progression, or to prevent the disorder from re-occurring once it has been initially eliminated (i.e., to prevent a relapse). The term “treatment”, as used herein, unless otherwise indicated, refers to the act of “treating”.

The term “therapeutically effective amount” as used herein refers to an amount that is effective, upon single or multiple dose administration to a subject (such as a human patient) in the prophylactic or therapeutic treatment of a disease, disorder or pathological condition as defined herein. For instance, said prophylactic or therapeutic effect is comparable to that of bortezomib, carfilzomib, lenalidomide, thalidomide, cyclophosphamide or any of the drugs generally used in the treatment of multiple myeloma.

The term “combination” or “combination therapy” as used throughout the specification, is meant to comprise the administration of the referred therapeutic agents to a subject suffering from cancer, in the same or separate pharmaceutical formulations, and at the same time or at different times. If the therapeutic agents are administered at different times they should be administered sufficiently close in time to provide for the combined effect (e.g. potentiating or synergistic response) to occur. The particular combination of therapies to employ in a combination regimen will take into account compatibility of the desired therapeutics and/or procedures and/or the desired therapeutic effect to be achieved. It will be appreciated that the therapies employed may achieve a desired effect for the same disorder (for example, anticancer effects), and/or they may achieve different effects (e.g., control of any adverse effects).

The term “resistance” as used herein refers to lack of response to a cancer treatment. It can be either “primary (de novo)”, when resistance occurs because of traits that a tumor has before treatment (i.e., some inherent characteristics of the cancer cells prevent the treatment's effectiveness), or “secondary (acquired)”, which occurs when tumors become resistant during treatment because of traits that tumor cells gain in response to the treatment.

The term “subject” as used herein refers to a mammalian subject. Preferably, it is selected from a human, companion animal, non-domestic livestock or zoo animal. For example, the subject may be selected from a human, dog, cat, cow, pig, sheep, horse, bear, and so on. In a preferred embodiment, said mammalian subject is a human subject.

The term “pharmaceutically acceptable salt” as used herein refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, trifluoroacetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, and ammonium. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

The term “prodrug” as used herein encompasses those derivatives that are converted in vivo to the compound of formula (I). The prodrug can hydrolyze, oxidize, or otherwise react under biological conditions to provide the compound of formula (I). Examples of prodrugs include, but are not limited to, derivatives that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Prodrugs can typically be prepared using well-known methods, such as those described by Burger “Medicinal Chemistry and Drug Discovery 6th ed. (Donald J. Abraham ed., 2001, Wiley) and “Design and Applications of Prodrugs” (H. Bundgaard ed., 1985, Harwood Academic Publishers).

### DETAILED DESCRIPTION OF THE INVENTION

The first aspect of the invention relates to a compound of formula (I):

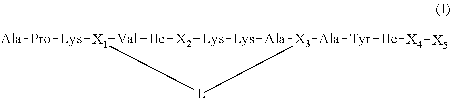

wherein X₂ is a non-polar amino acid, preferably selected from the group consisting of Leu and Phe; and

wherein X₄ is an amino acid, preferably Leu;

wherein X₅ is an amino acid, preferably Ser;

wherein X₁ and X₃ are independently selected and have formula (II):

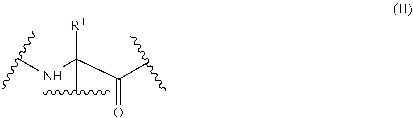

wherein R₁ is H or a monoradical selected from the group consisting of: (C₁-C₁₀)alkyl, (C₂-C₁₀)alkenyl, (C₂-C₁₀)alkynyl, (C₁-C₁₀)alkyl-O—(C₁-C₁₀)alkyl, (C₁-C₁₀)alkyl-C(═O)—(C₁-C₁₀)alkyl, (C₁-C₁₀)alkyl-O—C(O)—(C₁-C₁₀)alkyl, (C₁-C₁₀)alkyl-C(O)—NR₂—(C₁-C₁₀)alkyl, (C₁-C₁₀)alkyl-S—(C₁-C₁₀)alkyl, (C₁-C₁₀)alkyl-SR₃—(C₁-C₁₀)alkyl, (C₁-C₁₀)alkyl-S(═O)₂—(C₁-C₁₀)alkyl, (C₁-C₁₀)alkyl-S(═O)—(C₁-C₁₀)alkyl, (C₁-C₁₀)alkyl-O—S(═O)₂—O—(C₁-C₁₀)alkyl, (C₁-C₁₀)alkyl-NR₄—(C₁-C₁₀)alkyl; and
a (preferably known) ring system comprising from 3 to 14 carbon atoms, the system comprising from 1 to 3 rings, where:
   each one of the rings is saturated, partially unsaturated, or aromatic;
   the rings are isolated, partially or totally fused,
   each one of the members forming the (preferably known) ring system is selected from the group consisting of: —CH—, —CH₂—, —NH—, —N—, —SH—, —S—, and —O—; and
   the ring system is optionally substituted by one or more radicals independently selected from the group consisting of halogen, —OH, —NO₂, (C₁-C₁₀)alkyl, (C₁-C₁₀)haloalkyl, and (C₁-C₁₀)alkyl-O—; and
R₂, R₃ and R₄ are monoradicals independently selected from the group consisting of: hydrogen, (C₁-C₁₀)alkyl, (C₂-C₁₀)alkenyl, and (C₂-C₁₀)alkynyl; and
wherein L is a biradical bound to X₁ and X₃ via the alpha carbon, and is selected from the group consisting of: —O—, O—(C₁-C₁₀)alkyl-O—, O—(C₁-C₁₀)alkenyl-O—, C(═O), C(═O)NR₅, C(═O)O, NR₆, S—S—, S—(C₁-C₁₀)alkyl-S, S—(C₁-C₁₀)alkenyl-S— and a (preferably known) ring system consisting of one ring from 3 to 6 members, the ring:
   being saturated, partially unsaturated, or aromatic;
   each one of the members forming the (preferably known) ring system being selected from the group consisting of: —CH—, —CH₂—, —NH—, —N—, —SH—, —S—, and —O—; and
   the ring system being optionally substituted by one or more radicals independently selected from the group consisting of halogen, —OH, —NO₂, (C₁-C₁₀)alkyl, (C₁-C₁₀)haloalkyl, and (C₁-C₁₀)alkyl-O—; and
R₅ and R₆ are radicals independently selected from the group consisting of: —H and (C₁-C₁₀)alkyl (C₁-C₁₀)alkyl, (C₂-C₁₀)alkenyl, and (C₂-C₁₀)alkynyl;

for use in a method of treating a subject having multiple myeloma.

The term “compound of formula (I)” is intended here to cover any pharmaceutically acceptable salt or solvate (e.g. hydrate) thereof. It may further cover any prodrug or any other compound which directly or indirectly is capable of providing the compound of formula (I), and active metabolites of the compound of formula (I). Preferably, it relates to a compound of formula (I) or any pharmaceutically acceptable salt or solvate (e.g. hydrate) thereof.

The c-Myc protein is a transcription factor which plays an important role in the regulation of cell growth and differentiation, which aberrant overexpression is associated to carcinogenesis (Cole M D. Ann. Rev. Gen 1986, 20, 361-385; Henriksson M and Luscher B. Adv. Cancer Res. 1996, 68, 109-182; Marcu K B, Bossone S A and Patel A J. Ann. Rev. Biochem. 1992, 61, 809-860). c-Myc exhibits sequence-specific DNA binding when dimerized with its partner Max interacting through an helix-loop-helix zipper domain (Blackwood. E. M., and Eisenman. R. N., Science 1991, 251, 1211-1217; Blackwood, E. M., Lilscher. B., and Eisenman. R. N., Genes Dev. 1992, 6; 71-80; Blackwell. T. K. et al., Science 1990, 250, 1149-1151; Blackwell. T. K. et al., Mol. Cell. Biol. 1993, 13: 5216-5224). Without willing to be bound by theory, the compound of formula (I) is believed to exert its antiproliferative effects by preventing the formation of a complex between c-Myc and Max.

Preferably, R₁ of formula (II) is a monoradical selected from the group consisting of: H, (C₁-C₁₀)alkyl, (C₂-C₁₀)alkenyl, and (C₂-C₁₀)alkynyl, more preferably R, is H or a C₁-C₁₀ alkyl, more preferably R₁ is H or a C₁-C₄ alkyl, and even more preferably R, is H or —CH₃.

In preferred embodiments, X₁ and X₃ are Ala or Gly, X₂ is selected from Leu and Phe, X₄ is Leu and X₅ is Ser. Thus, in such preferred embodiments, the peptide sequence in the compound of formula (I) is selected from the group consisting of:

SEQ ID NO: 1 (Ala-Pro-Lys-Ala-Val-Ile-Leu-Lys-Lys-Ala-Ala-Ala-Tyr-11e-Leu-Ser)

SEQ ID NO: 2 (Ala-Pro-Lys-Ala-Val-Ile-Phe-Lys-Lys-Ala-Ala-Ala-Tyr-11e-Leu-Ser)

SEQ ID NO: 12 (Ala-Pro-Lys-Gly-Val-Ile-Leu-Lys-Lys-Ala-Gly-Ala-Tyr-11e-Leu-Ser)

Preferably, the peptide sequence is SEQ ID NO: 1.

Various ligands (L) may be used in the compound of formula (I) for peptide cross-linking between positions X₁ and X₃. Cross-linking systems (also referred as “staplers”) are known in the art and are typically used to maintain the alpha-helical structure of the peptide in solution and/or increase its stability in vivo. Such ligands have been described for instance in Schafmeister C E et al., Journal of the American Chemical Society 2000, 122 (24), 5891-5892; Walensky et al., Journal of Medicinal Chemistry 2014, 57(15), 6275-6288; Cromm et al., ACS Chemical Biology 2015, 10(6), 1362-1375; and De Araujo et al., Angew Chem Int Ed Engl. 2014, 53(27), 6965-9.

In preferred embodiments, L is a biradical having a straight-chain or branched C₃-C₃₀ hydrocarbon which may be interrupted once or more than once by one or more groups independently selected from: —O—, —S—, —SO—, NH—, —CO—, —NMe-, —NHCO—, —CONH—, arylene groups, heteroarylene groups, straight or branched C₁-C₆-alkylene groups and cyclic alkylene groups and 5-10 membered heterocyclic groups having up to 4 heteroatoms selected from the group consisting of N, O and S; optionally substituted by one or more substituents selected from the group consisting of halogen, —OH, —COOH, —NH₂, —NO₂, C₁-C₆ alkyl and C₁-C₆ alkenyl.

In a particular embodiment, L has the formula (IIIa):

—(CH₂)_{y}P—(CH₂)ₐ-(Q)_{b}-(CH₂)_{c}—(V)_{d}—(CH₂)_{z}— (IIIa)

wherein

P, Q and V are each independently selected from the group comprising O, S, NH, CONH, C(O)O, C₁-C₆ alkylene, arylene groups such as phenylene and heteroarylene groups such as triazolene, optionally substituted by an halogen;

y and z are integer values each independently selected from 1 to 10;

a and c are integer values each independently selected from 0 to 10; and

b and d are integer values each independently selected from 0 to 3.

In preferred embodiments, this ligand is selected from the group consisting of:

—(CH₂)_{y}—CH═CH—(CH₂)_{z}— (IIIb),

preferably wherein y and z are the same or different and are integer values selected from 1 to 10, including 1, 2, 3, 4, 5, 6, 7, 8, 9 and 10. Preferably y and z are integer values independently selected from 3 to 6, more preferably are integer values independently selected from 3 and 6;

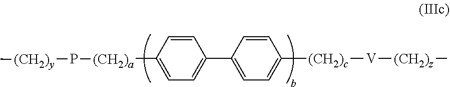

preferably wherein P and V are each independently selected from the group consisting of 0 and S; also preferably, y, and z are 1; preferably a is selected from 1 or from 6 to 10; preferably b is 0 or 1; and preferably c is 0 or 1;

Some particular embodiments of formula (IIIc) are:

—(CH₂)_{y}—P—(CH₂)ₐ—V—(CH₂)_{z}— (IIId1),

preferably wherein P and V are each independently selected from the group consisting of 0 and S; also preferably, y and z are 1, and a is selected from 6 to 10, preferably a is 8. In some preferred embodiments, the ligand of formula (IIId1) is selected from (IIId1.1) and (IIId1.2):

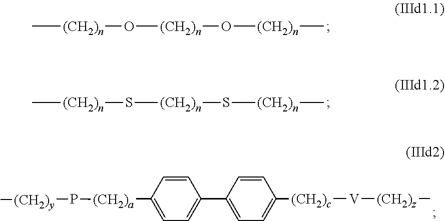

preferably wherein P and V are each independently selected from the group consisting of O and S; also preferably, y, a, c and z are 1. In some preferred embodiments, the ligand of formula (IIId2) is selected from (IIId2.1) and (IIId2.2):

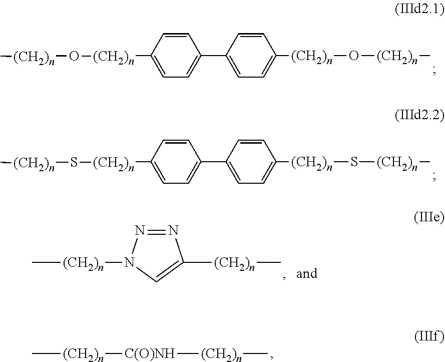

wherein in formulae (IIId1.1), (IIId1.2), (IIId2.1), (IIId2.2), (IIIe) and (IIIf) each n is an integer value independently selected from 1 to 10, including 1, 2, 3, 4, 5, 6, 7, 8, 9 and 10. In some preferred embodiments, each n is an integer value independently selected from 3 to 6. Preferably, each n is an integer value independently selected from 3 to 6 for ligands of formula (IIIe) or (IIIf). In other preferred embodiments, each n is independently selected from 1 or 2. Preferably, each n is 1 for ligands of formula (IIId).

In a particular embodiment, optionally in combination with one or more of the embodiments or features mentioned above or below, the compound of formula (I) for use according to the invention, is one wherein X₂ is selected from the group consisting of Leu and Phe; and wherein L is of formula (IIIb):

—(CH₂)_{y}—CH═CH—(CH₂)_{z}— (IIIb)

wherein y and z are the same or different and are integer values selected from 1 to 10, including 1, 2, 3, 4, 5, 6, 7, 8, 9 and 10. Preferably y and z are integer values independently selected from 3 to 6, more preferably are integer values independently selected from 3 and 6; also preferably X₁ and X₃ have formula (II) and R₁ is selected from —CH₃ or H.

In another particular embodiment, optionally in combination with one or more of the embodiments or features mentioned above or below, the compound of formula (I) for use according to the invention, is one wherein X₂ is selected from the group consisting of Leu and Phe; and wherein L is of formula (IIIc):

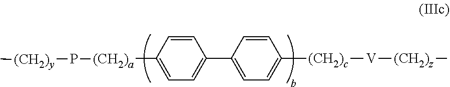

preferably wherein P and V are each independently selected from the group consisting of O and S; also preferably, y, and z are 1; preferably a is selected from 1 or from 6 to 10; preferably b is 0 or 1; preferably c is 0 or 1; also preferably X₁ and X₃ have formula (II) and R₁ is selected from —CH₃ or H.

In a further particular embodiment, optionally in combination with one or more of the embodiments or features mentioned above or below, the compound of formula (I) for use according to the invention, is one wherein X₂ is selected from the group consisting of Leu and Phe; and wherein L is of formula (IIId1):

—(CH₂)_{y}—P—(CH₂)ₐ—V—(CH₂)_{z}— (IIId1)

preferably wherein P and V are each independently selected from the group consisting of O and S; also preferably, y and z are 1 and a is selected from 6 to 10, preferably a is 8; also preferably X₁ and X₃ have formula (II) and R₁ is selected from —CH₃ or H.

In still a further particular embodiment, optionally in combination with one or more of the embodiments or features mentioned above or below, the compound of formula (I) for use according to the invention, is one wherein X₂ is selected from the group consisting of Leu and Phe; and wherein L is of formula (IIId2):

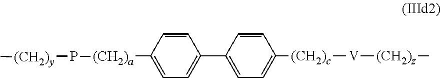

preferably wherein P and V are each independently selected from the group consisting of O and S; also preferably, y, a, c and z are 1; also preferably X₁ and X₃ have formula (II) and R, is selected from —CH₃ or H.

In a preferred embodiment, the compound of formula (I) for use according to the invention is selected from the group consisting of S09, S14, IDP-P1708160 and IDP-P1708161, with the formulas shown below, and combinations thereof:

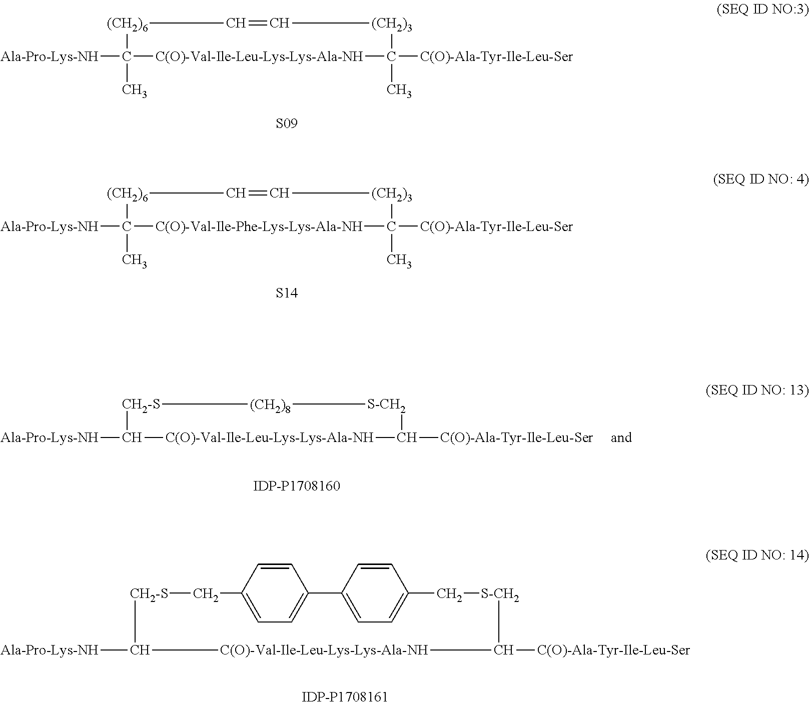

In a particular embodiment, the compound of formula (I) is S09.

A compound of formula (I) for use in accordance with the present invention may be prepared using Fmoc solid phase peptide chemistry. This process comprises the coupling, by condensation, of the carboxylic group or C-terminus of one amino acid with the amino group or N-terminus of another, this coupling reaction being repeated the number of times required until the desired peptide is obtained.

The process of preparation of the peptides may vary according to the nature of the L bridge, some illustrative non-limiting examples are as follows:

(1.a) the coupling, by condensation, of the corresponding amino acids of the peptide with a compound of formula (IV) and a compound of formula (V), which correspond to the amino acids referred as X1 and X3. Compounds (IV) and (V) will be those undergoing a subsequent cyclization step to generate the “L” bridge:

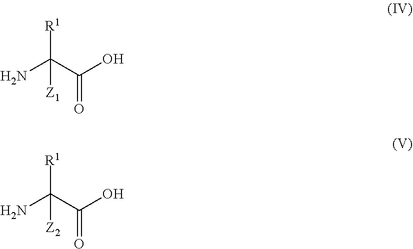

wherein R₁ is as defined above, Z₁ and Z₂ are the same or different and represent (C₂-C₁₀)alkenyl; and

(1.b) a cyclization step comprising the ring-closed (cf. Kim Young-Woo et al., “Synthesis of all-hydrocarbon stapled a-helical peptides by ring-closing olefin metathesis”, Nature Protocols, 2011, 6(6), p. 761-771; Scott J. M. et al., “Application of Ring-Closing Metathesis to the Synthesis of Rigidified Amino Acids and Peptides”, J. Am. Chem. Soc., 1996, v. 118 (40), pp 9606-9614) performed in solution with a Grubbs (I or II generation) catalyst; or, alternatively,

(2a) the coupling, by condensation, of the required amino acids, including a compound of formula (VI) and a compound of formula (VII), which correspond to the amino acids referred X1 and X3. Compounds (VI) and (VII) will be those undergoing a subsequent cyclization step to generate the “L” birradical”:

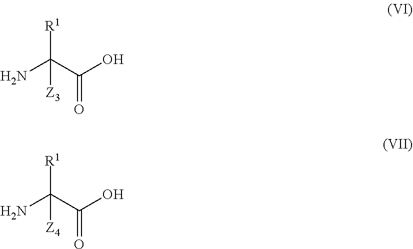

wherein R₁ is as defined above, Z₃ and Z₄ are the same or different, selected from the group consisting of: halogen —SH, —NHR₇, —OH, (C₂-C₁₀)alkyl-SH, (C₁-C₁₀)alkyl-OH, (C₁-C₁₀)alkyl-NHR₈, C(═O)OH, (C₁-C₁₀)C(═O)OH, OR₉, C(═O)-halogen, C(═O)—OR₁₀, preferably Z₃ is (C₁-C₁₀)C(═O)OH and Z₄ is (C₁-C₁₀)alkyl-NHR₈ or Z₃ is (C₁-C₁₀)alkyl-NHR₈ and Z₄ is (C₁-C₁₀)C(═O)OH;

where R₇ R₈ R₉ and R₁₀ are monoradicals independently selected from the group consisting of: hydrogen, (C₁-C₁₀)alkyl, (C₂-C₁₀)alkenyl, and (C₂-C₁₀)alkinyl; a (preferably known) ring system comprising from 3 to 14 carbon atoms, the system comprising from 1 to 3 rings, where:
each one of the rings is saturated, partially unsaturated, or aromatic;
the rings are isolated, partially or totally fused,
each one of the members forming the (preferably known) ring system is selected from the group consisting of: —CH—, —CH₂—, —NH—, —N—, —SH—, —S—, and —O—; and
the ring system is optionally substituted by one or more radicals independently selected from the group consisting of halogen, —OH, —NH₂, —SH, C(═O)-halogen (C₁-C₁₀)haloalkyl, and (C₁-C₁₀)alkyl-O—; and

(2b) a cyclization step comprising the coupling of Z₃ and Z₄ radicals; or, alternatively,

(3a) the coupling, by condensation, of the corresponding amino acids of the peptide with a compound of formula (VIII) and a compound of formula (IX), which correspond to the amino acids referred as X1 and X3. Compounds (VIII) and (IX) will be those undergoing a subsequent cyclization step to generate the “L” birradical”:

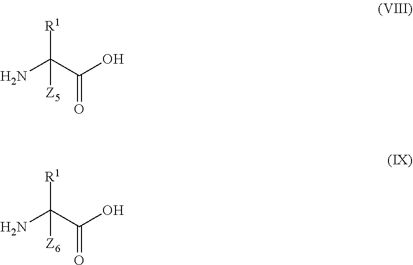

wherein R₁ is as defined above, one of Z₅ and Z₆ is (C₂-C₁₀)alkynyl and the other is (C₂-C₁₀)alkyl-N₃; and

(3.b) a cyclization step comprising the condensation of Z₅ and Z₆ radicals by well-known protocols such as the Cu(I)-mediated Huisgen 1,3-dipolar cycloaddition reaction (a.k.a. a “click” reaction) to generate a 1,4-substituted 1,2,3-triazole bridge (cf. Kolb H. C. et al., “The growing impact of click chemistry on drug discovery”, 2003, Drug Discov Today, 8(24):1128-1137); or, alternatively,

(4a) the coupling, by condensation, of the required amino acids, including a compound of formula (X) and a compound of formula (XI), which correspond to the amino acids referred X1 and X3. Compounds (X) and (XI) will be those undergoing a subsequent cyclization step to generate the “L” birradical”:

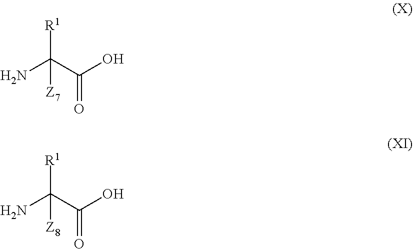

wherein R₁ is as described above (preferably H), and Z₇ is —(CH₂)_{y}—P and Z₈ is —(CH₂)_{z}—V as described above for formula (IIIc) (preferably Z₇ and Z₈ is —CH₂—S); and

(4b) a cyclization step comprising the coupling of Z₇ and Z₈ radicals with Br—(CH₂)ₐ-(biphenyl)_{b}-(CH₂)_{c}, wherein a, b and c are as described above for formula (IIIc), such as Br—(C₂-C₁₀)—Br or Br—CH₂-biphenyl-CH₂—Br by well-known protocols such as described in Doron C. Greenbaum et al. “Development of alpha-Helical Calpain Probes by Mimicking a Natural Protein-Protein Interaction”, JACS 2012).

In a preferred embodiment, a compound of formula (I) for use in accordance with the present invention is prepared using Fmoc solid phase peptide chemistry following the synthetic process described in the Examples.

The present invention also relates to the use of a compound of formula (I) in the manufacturing of a medicament for the treatment of multiple myeloma.

In addition, the present invention provides a method of treating multiple myeloma comprising administering to a subject in need of such treatment a therapeutically effective amount of a compound of formula (I).

Typically, a therapeutic treatment is administered to those patients which have been diagnosed with active multiple myeloma. For decades the diagnosis of active multiple myeloma required the presence of end-organ damage known as the CRAB criteria, including increased calcium level, renal dysfunction, anemia, and destructive bone lesions. The International Myeloma Working Group (IMWG) has recently reviewed the criteria for diagnosis of multiple myeloma (http://imwg.myeloma.org/international-myeloma-working-group-imwg-criteria-for-the-diagnosis-of-multiple-myeloma; NCCN Guidelines Insights, Multiple Myeloma, version 3. 2016, J Natl Compr Canc Netw 2016; 14(4):389-400) The updated criteria allow for treatment of patients who are at such high risk of progression to symptomatic disease that it is clear they would benefit from therapy and also potentially live longer if they were treated before serious organ damage occurred.

In the revised IMWG the presence of at least one of these markers is considered sufficient for diagnosis of active multiple myeloma, regardless of the presence or absence of symptoms or CRAB features. Each of these markers has been shown in two or more independent studies to be associated with an approximately 80% or higher risk of developing myeloma-related organ damage within two years. Accordingly, in a preferred embodiment, said subject has been diagnosed with active multiple myeloma. Preferably, a patient is diagnosed with active multiple myeloma when the following criteria are met:

Clonal bone marrow plasma cells ≥10% or biopsy-proven bony or extramedullary plasmacytoma; and

any one or more of the following myeloma-defining events:
Hypercalcemia: serum calcium >0.25 mmol/L (>1 mg/dL) higher than the upper limit of normal or >2.75 mmol/L (>11 mg/dL)
Renal insufficiency: creatinine clearance <40 mL per minute or serum creatinine >177 μmol/L (>2 mg/dL)
Anemia: hemoglobin >20 g/L below the lowest limit of normal, or a hemoglobin value <100 g/L
Bone lesions: one or more osteolytic lesion on skeletal radiography, CT, or PET/CT. If bone marrow has <10% clonal plasma cells, more than one bone lesion is required to distinguish from solitary plasmacytoma with minimal marrow involvement
60% or greater clonal plasma cells on bone marrow examination
Serum involved/uninvolved free light chain ratio of 100 or greater, provided the absolute level of the involved light chain is at least 100 mg/L (a patient's involved free light chain either kappa or lambda is the one that is above the normal reference range; the uninvolved free light chain is the one that is typically in, or below, the normal range)
More than one focal lesion on MRI that is at least 5 mm or greater in size.

Depending on the development stage of the disease, anticancer effects of the methods of treatment of the present invention include, but are not limited to, inhibition of tumor growth, tumor growth delay, regression of tumor, shrinkage of tumor, reduction of tumor size and/or tumor markers, increased time to regrowth of tumor on cessation of treatment, and slowing of disease progression. In a particular embodiment, the methods of treatment of the invention are suited for human patients, especially those who are resistant, relapsing or refractory to previous treatment (e.g. chemotherapy, targeted therapy or corticosteroids). First line therapy is also envisaged.

In a particular embodiment, optionally in combination with one or more of the embodiments or features described above or below, said multiple myeloma is refractory or relapsing multiple myeloma, in other words myeloma cells are found to be or have become resistant to a treatment. Lack of response to the treatment may be identified clinically as progressive disease or clinical relapse.

Progressive disease in multiple myeloma is generally found when one or more of the following has occurred: at least 25% increase in the amount of M-proteins in the blood or urine, a 25% increase in the number of plasma cells in the bone marrow, an increase in the size or number of bone lesions, or an increase in the calcium levels not explained by other conditions. Clinical relapse is typically found when one or more of the following has occurred: direct signs of cancer growth, signs of organ damage, an increase in the number or size (at least 50% larger) of plasmacytomas or bone lesions, increased calcium levels, and increase in creatinine levels in blood, or a decrease in the number of red blood cells (NCCN Guidelines for Patients®, Multiple Myeloma, version 1.2016).

In a preferred embodiment, said multiple myeloma is resistant to a drug selected from the group consisting of an alkylating agent (preferably, a nitrogen mustard), a corticosteroid and an anthracycline.

The alkylating agents are compounds that react with electron-rich atoms in biologic molecules to form covalent bonds. Traditionally, these agents are divided into two types: those that react directly with biologic molecules and those that form a reactive intermediate, which then reacts with the biologic molecules. This chemotherapeutic group includes but is not limited to nitrogen mustards (e.g., cyclophosphamide, chlormethine, uramustine, melphalan, chlorambucil, ifosfamide, and bendamustine), nitrosoureas (e.g., armustine, lomustine, and streptozocin) and alkyl sulfonates (e.g., busulfan).

Corticosteroids play an important role in treatment of multiple myeloma and have both anti-inflammatory and anti-myeloma effects. Non-limiting examples of this therapeutic group are dexamethasone, prednisolone and methylprednisolone.

Anthracyclines, also referred as anthracycline antibiotics, include daunorubicin, doxorubicin and analogs thereof, such as epirubicin, idarubicin, mitoxantrone, pixantrone, and valrubicin.

In a preferred embodiment, said multiple myeloma is resistant to a drug selected from the group consisting of melphalan, dexamethasone and doxorubicin. This may occur for instance when one of said drugs has been used in said subject in a prior treatment alone or in combination with other drugs.

Pharmaceutical Composition

The invention also provides a pharmaceutical composition comprising a compound of formula (I), and a pharmaceutically acceptable carrier or excipient, for use in a method of treating a subject having multiple myeloma.

The expression “pharmaceutically acceptable excipient or carrier” refers to pharmaceutically acceptable materials, compositions or vehicles. Each component must be pharmaceutically acceptable in the sense of being compatible with the other ingredients of the pharmaceutical composition. It must also be suitable for use in contact with the tissue or organ of humans and animals without excessive toxicity, irritation, allergic response, immunogenicity or other problems or complications commensurate with a reasonable benefit/risk ratio. Likewise, the term “veterinary acceptable” means suitable for use in contact with a non-human animal. Examples of suitable pharmaceutically acceptable excipients are solvents, dispersion media, diluents, or other liquid vehicles, dispersion or suspension aids, surface active agents, isotonic agents, thickening or emulsifying agents, preservatives, solid binders, lubricants and the like. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

The formulations of the pharmaceutical compositions described herein may be prepared by any method known or hereafter developed in the art of pharmacology. In general, such preparatory methods include the step of bringing the active ingredient into association with a excipient and/or one or more other accessory ingredients, and then, if necessary and/or desirable, shaping and/or packaging the product into a desired single- or multi-dose unit.

A pharmaceutical composition for use in a method of treatment of the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

The relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered.

Pharmaceutically or veterinary acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in the inventive formulations. Excipients such as cocoa butter and suppository waxes, coloring agents, coating agents, sweetening, flavoring, and perfuming agents can be present in the composition, according to the judgment of the formulator.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked polyvinylpyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters {e.g., polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g., polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g., Cremophor), polyoxyethylene ethers, (e.g., polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof.

Exemplary binding agents include, but are not limited to, starch (e.g., cornstarch and starch paste); gelatin; sugars (e.g., sucrose, glucose, dextrose, dextrin, molasses, lactose, lactitol, mannitol); natural and synthetic gums (e.g., acacia, sodium alginate, extract of Irish moss, panwar gum, ghatti gum, mucilage of isapol husks, carboxymethylcellulose, methylcellulose, ethylcellulose, hyd roxyethylcellulose, hydroxypropyl cellulose, hydroxypropyl methylcellulose, microcrystalline cellulose, cellulose acetate, polyvinylpyrrolidone), magnesium aluminum silicate (Veegum), and larch arabogalactan); alginates; polyethylene oxide; polyethylene glycol; inorganic calcium salts; silicic acid; polymethacrylates; waxes; water; alcohol; and combinations thereof.

Exemplary preservatives may include antioxidants, chelating agents, antimicrobial preservatives, antifungal preservatives, alcohol preservatives, acidic preservatives, and other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and sorbic acid. Exemplary alcohol preservatives include, but are not limited to, ethanol, polyethylene glycol, phenol, phenolic compounds, bisphenol, chlorobutanol, hydroxybenzoate, and phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Liquid dosage forms for oral and parenteral administration include, but are not limited to, pharmaceutically or veterinary acceptable liposomes emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates of the invention are mixed with solubilizing agents such as polyethoxylated castor oil (e.g. CREMOPHOR™), alcohols, oils, modified oils, glycols, polysorbates, cyclodextrins, polymers, and combinations thereof.

Injectable preparations, for example, sterile injectable aqueous or oleaginous suspensions may be formulated according to the known art using suitable dispersing or wetting agents and suspending agents. The sterile injectable preparation may be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. Alternatively, the preparation can be in the form of liposomes.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Solid dosage forms for oral administration include capsules, tablets, pills, powders, and granules. In such solid dosage forms, the active ingredient is mixed with at least one inert, pharmaceutically acceptable excipient such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may comprise buffering agents.

Solid compositions of a similar type may be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polethylene glycols and the like.

A compound of formula (I) for use in the methods of treatment of the invention can be in micro-encapsulated form with one or more excipients as noted above, for instance formulated in liposomes. The solid dosage forms of tablets, dragees, capsules, pills, and granules can be prepared with coatings and shells such as enteric coatings, release controlling coatings and other coatings well known in the pharmaceutical formulating art. In such solid dosage forms the active ingredient may be admixed with at least one inert diluent such as sucrose, lactose or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes.

The compound of formula (I) for use in a method of treatment of the invention is typically formulated as a pharmaceutical composition to be compatible with its intended route of administration. Methods to accomplish the administration are known to those of ordinary skill in the art. This includes, for example, injection or infusion, by parenteral routes such as intravenous, intravascular, intraarterial, subcutaneous, intramuscular, intraperitoneal, intraventricular, intraepidural, or others as well as oral, sublingual, nasal, ophthalmic, rectal, transdermal or topical. Sustained release administration is also specifically contemplated, e.g., as depot injections or erodible implants. Localized delivery is particularly contemplated, e.g., as delivery via a catheter to one or more arteries, such as the renal artery or a vessel supplying a localized site of interest.

In a particular embodiment, the compound of formula (I) is administered/formulated for administration by the oral, sublingual, transdermal or parenteral route. Preferably, the compound of formula (I) is administered/formulated for administration by the parenteral route, which includes intravenous, intramuscular, intraperitoneal, intrapreural or intravenous administration, preferably for the intravenous administration. Administration by the intravascular route is carried out using devices well known in the art, which are used to administer fluids from a container to a patient's vascular system through a needle or catheter inserted into a vein. The device may include the needle or catheter, tubing, a flow regulator, a drip chamber, an infusion line filter, an I.V. set stopcock, fluid delivery tubing, connectors between parts of the set, a side tube with a cap to serve as an injection site, and a hollow spike to penetrate and connect the tubing to an I.V. bag or other infusion fluid container.

Preferably, said composition is in a form suitable for intravascular administration. In a preferred embodiment, said composition is an aqueous composition, more preferably a stable aqueous composition. As used herein, a “stable composition” may refer to a formulation in which the active ingredient therein essentially retains its physical stability and/or chemical stability and/or biological activity upon storage.

Some embodiments of such compositions may be provided by lyophilised formulations. Said lyophilised formulations can be reconstituted and diluted to give a composition of this invention in the form of a solution ready for intravascular injection. Preferably, said lyophilised formulations are presented in single dose containers.

Reconstituted embodiments of the present invention can further be diluted if so desired. This further dilution is preferably carried out with an aqueous diluent as described herein. The reconstituted solution will be diluted depending on the concentration in the reconstituted solution and the desired concentration in the diluted solution.

A compound of formula (I) can be administered a single time. It may also be administered regularly throughout the course of the method of treatment, for example, one, two, three, four, or more times a day, every other day, weekly, bi-weekly, every three weeks or monthly.

In a particular embodiment, optionally in combination with any of the features or embodiments described above or below, a compound of formula (I) is administered daily, preferably once or twice per day.

In another particular embodiment, optionally in combination with any of the features or embodiments described above or below, the compound of formula (I) is administered twice or three times per week. For instance, it may be administered at days 1 to 7; at days 1, 3, and 5; or at days 1 and 4 of each week of a treatment cycle.

The duration and number of cycles is not particularly limited. Cycles may be repeated for instance until maximal response, disease progression or unacceptable toxicity is achieved. Also, cycles may be repeated every 3 or 4 weeks for 3-4 cycles.

A compound of formula (I) may also be administered continuously to the subject (e.g, intravenously or by release from an implant, pump, sustained release formulation, etc.).

The dosage to be administered can depend on multiple factors, including the type and severity of the disease and/or on the characteristics of the subject, such as general health, age, sex, body weight and tolerance to drugs and should be adjusted, as needed, according to individual need and professional judgment. The dosage may also vary depending upon factors, such as route of administration, treatment regime, target site, or other therapies administered. The skilled artisan will be able to determine appropriate doses depending on these and other factors. A prophylactic or therapeutically effective amount may include, but is not limited to, dosage ranges of about 0.001 mg/kg to about 100 mg/kg of body weight, preferably about 0.01 mg/kg to about 10 mg/kg, more preferably, about 0.1 mg/kg to about 1 mg/kg.

In a particular embodiment, optionally in combination with any of the features or embodiments described above or below, the compound of formula (I) is administered by the parenteral route of administration (preferably intravenously) at a dosage of about 0.1 to about 1 mg/kg of body weight, preferably of about 0.25 to about 0.5 mg/kg of body weight, more preferably of about 0.30 to about 0.35 mg/kg of body weight for a human adult and of about 0.45 to about 0.5 mg/kg of body weight for a human child.

A compound of formula (I) may be administered alone (as a single agent) or in combination with another drug.

Combination Therapy

The invention also relates to a compound of formula (I), or the pharmaceutical composition comprising the same, for use in a method of treatment of the invention wherein said treatment comprises the administration of a compound of formula (I) in combination with another drug. Each agent may be administered at a dose and/or on a time schedule generally used for that agent as single agent or in combination therapies. Dosage and administration regimens for the compound of formula (I) have been described herein.

This other drug may be another anticancer drug, such as chemotherapeutic agents, targeted therapies or corticosteroids usually used in the therapeutic treatment of multiple myeloma.

This other drug may also be an adjunctive treatment. This may include supportive care to manage the symptoms of myeloma and side effects of myeloma treatment. This includes for instance treatments for bone damage (e.g. bisphosphonates), kidney damage (plasmapheresis, administration of intravascular fluids), anemia (e.g. erythropoietin), infections (e.g. immunoglobulins), thrombosis (e.g. heparin), etc.

Additional details on the existing primary and adjunctive treatment regimens for multiple myeloma may be found for instance in Devita, Hellman, and Rosenberg's cancer: principles & practice of oncology, 10 edition, December 2014, Wolters Kluwer; and the NCCN guidelines insights multiple myeloma, version 3.2016 (J Natl Compr Canc Netw 2016; 14(4):389-400), Multiple Myeloma, version 3.2017 (J Natl Compr Canc Netw 2017; 15:230-269).

In one embodiment, optionally in combination with any of the embodiments or features described above or below, the peptides of the invention are administered in combination with one or more anti-cancer agents. The drugs may be administered in double, triple, quadruple combinations or in combinations comprising a higher number of agents. An anti-cancer agent may be, for instance, methotrexate, vincristine, adriamycin, cisplatin, non-sugar containing chloroethylnitrosoureas, 5-fluorouracil, mitomycin C, bleomycin, doxorubicin, dacarbazine, taxol, fragyline, Meglamine GLA, valrubicin, carmustaine and poliferposan, MM1270, BAY 12-9566, RAS farnesyl transferase inhibitor, farnesyl transferase inhibitor, MMP, MTAILY231514, LY264618/Lometexol, Glamolec, Cl-994, TNP-470, Hycamtin/Topotecan, PKC412, Valspodar/PSC833, Novantrone/Mitroxantrone, Metaret/Suramin, Batimastat, E7070, BCH-4556, CS-682, 9-AC, AG3340, AG3433, Incel/VX-710, VX-853, ZD0101, IS1641, ODN 698, TA 2516/Marmistat, BB2516/Marmistat, CDP 845, D2163, PD183805, DX895 if, Lemonal DP 2202, FK 317, Picibanil/OK-432, AD 32/Valrubicin, Metastron/strontium derivative, Temodal/Temozolomide, Evacet/liposomal doxorubicin, Yewtaxan/Paclitaxel, Taxol/Paclitaxel, Xeload/Capecitabine, Furtulon/Doxifluridine, Cyclopax/oral paclitaxel, Oral Taxoid, SPU-077/Cisplatin, HMR 1275/Flavopiridol, CP-358 (774)/EGFR, CP-609 (754)/RAS oncogene inhibitor, BMS-182751/oral platinum, UFT (Tegafur/Uracil), Ergamisol/Levamisole, Eniluracil/776C_{85/5}FU enhancer, Campto/Levamisole, Camptosar/Irinotecan, Tumodex/Ralitrexed, Leustatin/Cladribine, Paxex/Paclitaxel, Doxil/liposomal doxorubicin, Caelyx/liposomal doxorubicin, Fludara/Fludarabine, Pharmarubicin/Epirubicin, DepoCyt, ZD1839, LU 79553/Bis-Naphtalimide, LU 103793/Dolastain, Caetyx/liposomal doxorubicin, Gemzar/Gemcitabine, ZD 0473/Anormed, YM 116, iodine seeds, CDK4 and CDK2 inhibitors, PARP inhibitors, D4809/Dexifosamide, Ifes/Mesnex/Ifosamide, Vumon/Teniposide, Paraplatin/Carboplatin, Plantinol/cisplatin, Vepeside/Etoposide, ZD 9331, Taxotere/Docetaxel, prodrug of guanine arabinoside, Taxane Analog, nitrosoureas, alkylating agents such as melphelan and cyclophosphamide, Aminoglutethimide, Asparaginase, Busulfan, Carboplatin, Chlorombucil, Cytarabine HCl, Dactinomycin, Daunorubicin HCl, Estramustine phosphate sodium, Etoposide (VP16-213), Floxuridine, Fluorouracil (5-FU), Flutamide, Hydroxyurea (hydroxycarbamide), Ifosfamide, Interferon Alfa-2a, Alfa-2b, Leuprolide acetate (LHRH-releasing factor analogue), Lomustine (CCNU), Mechlorethamine HCl (nitrogen mustard), Mercaptopurine, Mesna, Mitotane (o.p-DDD), Mitoxantrone HCl, Octreotide, Plicamycin, Procarbazine HCl, Streptozocin, Tamoxifen citrate, Thioguanine, Thiotepa, Vinblastine sulfate, Amsacrine (m-AMSA), Azacitidine, Erthropoietin, Hexamethylmelamine (HMM), Interleukin 2, Mitoguazone (methyl-GAG; methyl glyoxal bis-guanylhydrazone; MGBG), Pentostatin (2′-deoxycoformycin), Semustine (methyl-CCNU), Teniposide (VM-26) or Vindesine sulfate, signal transduction inhibitors (such as MEK, BRAF, AKT, her2, mTOR, and PI3K inhibitors), but it is not so limited.

In a particular embodiment, optionally in combination with one or more of the embodiments or features described above or below, said other drug is selected from the group consisting of an alkylating agent, a corticosteroid a proteasome inhibitor, and combinations thereof.

The compound of formula (I) may be combined with an alkylating agent. Examples of alkylating agents have been provided herein above. Preferably, said alkylating agent is selected from the group consisting of nitrogen mustards, nitrosoureas and alkyl sulfonates.

More preferably said alkylating agent is a nitrogen mustard, even more preferably cyclophosphamide.

Cyclophosphamide has been used as single agent in high dose (600 mg/m2) IV over 60 minutes for 4 days. Cycles can be repeated every 4 weeks for 2 cycles, then every 3 months until maximal response, disease progression, or unacceptable toxicity.

An example of combined cyclophosphamide treatment is the combination with bortezomib and dexamethasone, where cyclophosphamide is administered on days 1, 8, 15 and 22 at a dose of 300 mg/m²/day orally and cycles are repeated every 4 weeks for 3-4 cycles.

Cyclophosphamide may be used in combination with a peptide of formula (I) at 600 mg/m² or in a lower dose, such as 500 mg/m², 400 mg/m², 300 mg/m², 200 mg/m² or 100 mg/m², parenterally or orally, preferably at a dose of about 300 mg/m²/day. At any of these dosages may for instance be used according to the above treatment regimens.

It may further be combined with a corticosteroid. Preferably, said corticosteroid is selected from the group consisting of dexamethasone, prednisolone and methylprednisolone, more preferably is dexamethasone.

Some examples of clinical use of dexamethasone in combination are provided below:
(i) in combination with bortezomib: days 1-4 (all cycles) and 9-12 (cycles 1 and 2) at 40 mg orally daily; or days 1-2, 4-5, 8-9, and 11-12 at 20 mg orally daily, repeat cycle every 3 weeks for 3-4 cycles.
(ii) combination with bortezomib and cyclophosphamide: days 1-4, 9-12 and 17-20 at 40 mg orally daily, repeat cycle every 4 weeks for 3-4 cycles;
(iii) in combination with doxorubicin and bortezomib: days 1-4, 9-12, and 17-20 at 40 mg orally daily for cycle 1, and on days 1-4 for cycles 2-4, repeat cycle every 3 weeks for 3-4 cycles.

Accordingly, for instance, dexamethasone may be used in combination with a compound of formula (I) at a dosage from 20 to 40 mg orally daily during days 1-4, 9-12, and 17-20, and cycles may be repeated every 3 or 4 weeks for 3-4 cycles. It may also be used at the above specified dosages and regimens.

Inhibition of proteasome function has emerged as a powerful strategy for anti-cancer therapy (Crawford et al., J Cell Commun Signal. 2011, 5(2): 101-110). The compound of formula (I) may also be combined with a proteasome inhibitor. Said proteasome inhibitor is preferably selected from the group consisting of bortezomib, carfilzomib and ixazomib, preferably is bortezomib. In addition said proteasome inhibitor (e.g. bortezomib) may additionally be combined for instance with doxorubicin, thalidomide, melphalan, dexamethasone, and lenalidomide, with which has generally been successfully combined without increased toxicity.

Bortezomib may be used in combination with a compound of formula (I) on days 1, 4, 8, and 11 at 1.3 mg/m2 IV push over 3-5 seconds or subcutaneous (SC). Cycles may be repeated every 3 or 4 weeks for 3-4 cycles.

In another particular embodiment, optionally in combination with one or more of the embodiments or features described above or below, said method of treatment comprises the administration of a compound of formula (I), a corticosteroid and a drug selected from the group consisting of an alkylating agent, a proteasome inhibitor, and combinations thereof.

Preferably, said corticosteroid is dexamethasone. In a preferred embodiment, the combination therapy comprises a compound of formula (I) and dexamethasone, preferably it comprises a compound of formula (I), dexamethasone and a compound selected from either bortezomib or cyclophosphamide.

In another particular embodiment, optionally in combination with one or more of the embodiments or features described above or below, said combination treatment is selected from the group consisting of or comprising:
the compound of formula (I)+bortezomib;
the compound of formula (I)+cyclophosphamide;
the compound of formula (I)+dexamethasone;
the compound of formula (I)+bortezomib+dexamethasone; and
the compound of formula (I)+cyclophosphamide+dexamethasone.

Preferred dosage & administration regimens are as described above.

In a particular embodiment, optionally in combination with one or more of the embodiments or features described above or below, the compound of formula (I) is administered at days 1 to 7 of each week of a treatment cycle, preferably once per day, and the other anticancer drug is administered at days 1 and 4 of each week of a treatment cycle, preferably once per day. The treatment may last 3-4 weeks and cycles are preferably repeated every 3-4 weeks.

In another particular embodiment, optionally in combination with one or more of the embodiments or features described above or below, the compound of formula (I) is administered at days 1, 3, and 5 of each week of a treatment cycle, preferably once per day, and the other anticancer drug is administered at days 1 and 4 of each week of a treatment cycle, preferably once per day. The treatment may last 3-4 weeks and cycles are preferably repeated every 3-4 weeks.

In a further particular embodiment, optionally in combination with one or more of the embodiments or features described above or below, the compound of formula (I) is administered at days 1 and 4 of each week of a treatment cycle, preferably once per day, and the other anticancer drug is administered at days 1 and 4 of each week of a treatment cycle, preferably once per day. The treatment may last 3-4 weeks and cycles are preferably repeated every 3-4 weeks.

Particular and preferred embodiments of the compound of formula (I) have been described herein above. Preferably, the compound of formula (I) is selected from the group consisting of S09, S14 and combinations thereof, more preferably the compound of formula (I) is S09.

A compound of formula (I) and said other drug, preferably another anticancer drug, can be administered in the same or separate pharmaceutical compositions, and at the same time (simultaneously) or at different times (a compound of formula (I) is administered before or after said other drug). Further details on the administration schedules of a combination therapy are provided above.

In a particular embodiment, administration of a compound of formula (I) is simultaneous to the administration of said other drug, as part of the same or separate compositions. In another particular embodiment, administration of a compound of formula (I) is sequential (prior to or subsequent) to the administration of said other drug.

The invention is also directed to the use of a compound of formula (I) for the manufacture of a medicament for an effective treatment of cancer by combination therapy employing a compound of formula (I) with another drug as described herein.

It is further directed to a method of treating cancer comprising administering to a patient in need of such treatment a therapeutically effective amount of a compound of formula (I), in combination with a therapeutically effective amount of another drug as described herein.

The invention also provides a pharmaceutical composition comprising a compound of formula (I), another drug, and a pharmaceutically acceptable carrier or excipient, for use in combination in a method of treating multiple myeloma as described herein.

The invention further provides a kit for use in a method of treating multiple myeloma as described herein, which comprises: a pharmaceutical composition comprising a compound of formula (I), and a pharmaceutical composition comprising another drug; and optionally, instructions for use of both drugs in combination in a method of treating multiple myeloma as described herein.

Items of the Invention
1. A compound of formula (I):

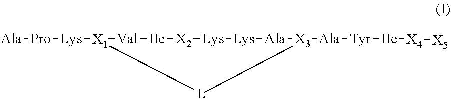

wherein X₂ is a non-polar amino acid, preferably selected from the group consisting of Leu and Phe; and

wherein X₄ is an amino acid, preferably Leu;

wherein X₅ is an amino acid, preferably Ser;

wherein X₁ and X₃ are independently selected and have formula (II):

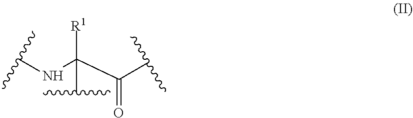

wherein R₁ is H or a monoradical selected from the group consisting of: (C₁-C₁₀)alkyl, (C₂-C₁₀)alkenyl, (C₂-C₁₀)alkynyl, (C₁-C₁₀)alkyl-O—(C₁-C₁₀)alkyl, (C₁-C₁₀)alkyl-C(═O)—(C₁-C₁₀)alkyl, (C₁-C₁₀)alkyl-O—C(O)—(C₁-C₁₀)alkyl, (C₁-C₁₀)alkyl-C(O)—NR₂—(C₁-C₁₀)alkyl, (C₁-C₁₀)alkyl-S—(C₁-C₁₀)alkyl, (C₁-C₁₀)alkyl-SR₃—(C₁-C₁₀)alkyl, (C₁-C₁₀)alkyl-S(═O)₂—(C₁-C₁₀)alkyl, (C₁-C₁₀)alkyl-S(═O)—(C₁-C₁₀)alkyl, (C₁-C₁₀)alkyl-O—S(═O)₂—O—(C₁-C₁₀)alkyl, (C₁-C₁₀)alkyl-NR₄—(C₁-C₁₀)alkyl; and
a (preferably known) ring system comprising from 3 to 14 carbon atoms, the system comprising from 1 to 3 rings, where:
   each one of the rings is saturated, partially unsaturated, or aromatic;
   the rings are isolated, partially or totally fused,
   each one of the members forming the (preferably known) ring system is selected from the group consisting of: —CH—, —CH₂—, —NH—, —N—, —SH—, —S—, and —O—; and
   the ring system is optionally substituted by one or more radicals independently selected from the group consisting of halogen, —OH, —NO₂, (C₁-C₁₀)alkyl, (C₁-C₁₀)haloalkyl, and (C₁-C₁₀)alkyl-O—; and
R₂, R₃ and R₄ are monoradicals independently selected from the group consisting of: hydrogen, (C₁-C₁₀)alkyl, (C₂-C₁₀)alkenyl, and (C₂-C₁₀)alkynyl; and
wherein L is a biradical bound to X₁ and X₃ via the alpha carbon, and is selected from the group consisting of: —O—, O—(C₁-C₁₀)alkyl-O—, O—(C₁-C₁₀)alkenyl-O—, C(═O), C(═O)NR₅, C(═O)O, NR₆, S—S—, S—(C₁-C₁₀)alkyl-S, S—(C₁-C₁₀)alkenyl-S— and a (preferably known) ring system consisting of one ring from 3 to 6 members, the ring:
   being saturated, partially unsaturated, or aromatic;
   each one of the members forming the (preferably known) ring system being selected from the group consisting of: —CH—, —CH₂—, —NH—, —N—, —SH—, —S—, and —O—; and
   the ring system being optionally substituted by one or more radicals independently selected from the group consisting of halogen, —OH, —NO₂, (C₁-C₁₀)alkyl, (C₁-C₁₀)haloalkyl, and (C₁-C₁₀)alkyl-O—; and
R₅ and R₆ are radicals independently selected from the group consisting of: —H and (C₁-C₁₀)alkyl (C₁-C₁₀)alkyl, (C₂-C₁₀)alkenyl, and (C₂-C₁₀)alkynyl;
for use in a method of treating a subject having multiple myeloma.
2. The compound of formula (I) for use in a method of treatment according to item 1, wherein X2 is selected from the group consisting of Leu and Phe; and wherein L is of formula (IIIb):

—(CH₂)_{y}—CH═CH—(CH₂)_{z}— (IIIb)
wherein y and z are the same or different and are integer values selected from 1 to 10, preferably are independently selected from 3 to 6, more preferably are independently selected from 3 and 6.
3. The compound of formula (I) for use in a method of treatment according to any of items 1 or 2, wherein said compound is selected from the group consisting of S09 (SEQ ID NO: 3), S14 (SEQ ID NO: 4), and combinations thereof.
4. The compound of formula (I) for use in a method of treatment according to any of items 1 to 3, wherein said multiple myeloma is resistant, refractory or relapsing multiple myeloma, preferably wherein said multiple myeloma is resistant, refractory or relapsing to a previous treatment.
5. The compound of formula (I) for use in a method of treatment according to item 4, wherein said multiple myeloma is resistant to a drug selected from the group consisting of an alkylating agent, a corticosteroid and an anthracycline.
6. The compound of formula (I) for use in a method of treatment according to any of items 4 or 5, wherein said multiple myeloma is resistant to a drug selected from the group consisting of melphalan, dexamethasone and doxorubicin.
7. The compound of formula (I) for use in a method of treatment according to any of items 1 to 6, wherein said treatment comprises the administration of a compound of formula (I) in combination with another drug.
8. The compound of formula (I) for use in a method of treatment according to item 7, wherein said other drug is selected from the group consisting of an alkylating agent, a corticosteroid, a proteasome inhibitor, and combinations thereof.
9. The compound of formula (I) for use in a method of treatment according to item 8, wherein said treatment comprises the administration of a compound of formula (I), a corticosteroid and a drug selected from the group consisting of an alkylating agent, a proteasome inhibitor, and combinations thereof.
10. The compound of formula (I) for use in a method of treatment according to any of items 8 or 9, wherein said alkylating agent is selected from the group consisting of nitrogen mustards, nitrosoureas and alkyl sulfonates, preferably is a nitrogen mustard, more preferably is cyclophosphamide.
11. The compound of formula (I) for use in a method of treatment according to any of items 8 to 10, wherein said corticosteroid is selected from the group consisting of dexamethasone, prednisolone and methylprednisolone, preferably is dexamethasone.
12. The compound of formula (I) for use in a method of treatment according to any of items 8 to 11, wherein said proteasome inhibitor is selected from the group consisting of bortezomib, carfilzomib and ixazomib, preferably is bortezomib.
13. The compound of formula (I) for use in a method of treatment according to item 7, wherein the compound of formula (I) is used in a combination treatment selected from the group consisting of:
   the compound of formula (I)+bortezomib;
   the compound of formula (I)+cyclophosphamide;
   the compound of formula (I)+dexamethasone;
   the compound of formula (I)+bortezomib+dexamethasone; and
   the compound of formula (I)+cyclophosphamide+dexamethasone.
14. The compound of formula (I) for use in a method of treatment according to any of items 7 to 13, wherein the compound of formula (I) is administered at days 1 to 7 of each week of a treatment cycle, preferably once or twice per day, and the other anticancer drug is administered at days 1 and 4 of each week of a treatment cycle, preferably once per day.
15. The compound of formula (I) for use in a method of treatment according to any of items 7 to 13, wherein the compound of formula (I) is administered at days 1, 3, and 5 of each week of a treatment cycle, preferably once per day, and the other anticancer drug is administered at days 1 and 4 of each week of a treatment cycle, preferably once per day.
16. The compound of formula (I) for use in a method of treatment according to any of items 7 to 13, wherein the compound of formula (I) is administered at days 1 and 4 of each week of a treatment cycle, preferably once per day, and the other anticancer drug is administered at days 1 and 4 of each week of a treatment cycle, preferably once per day.
17. The compound of formula (I) for use in a method of treatment according to any of items 1 to 16, wherein the treatment cycle lasts 3-4 weeks and cycles are repeated every 3-4 weeks.
18. The compound of formula (I) for use in a method of treatment according to any of items 1 to 17, wherein the compound of formula (I) is formulated to be administered at a dosage from 0.1 mg/kg to 1 mg/kg, preferably from 0.25 mg/kg to 0.5 mg/kg.
19. The compound of formula (I) for use in a method of treatment according to any of items 1 to 18, wherein the compound of formula (I) is formulated for parenteral administration, preferably for intravenous, intramuscular, intraperitoneal, intrapreural or intravenous administration, more preferably for intravenous administration.
20. The compound of formula (I) for use in a method of treatment according to any of items 1 to 19, wherein said subject is a human.
21. A pharmaceutical composition comprising the compound of formula (I) as defined in any of items 1 to 3, and a pharmaceutically acceptable carrier or excipient, for use in a method of treatment according to any of items 1 to 20.
22. A pharmaceutical composition comprising a compound of formula (I) as defined in any of items 1 to 3, another drug, and a pharmaceutically acceptable carrier or excipient, for use in a method of treatment according to any of items 7 to 20.
23. A kit for use in a method of treatment according to any of items 7 to 20, which comprises: a pharmaceutical composition comprising a compound of formula (I) as defined in any of items 1 to 3, and a pharmaceutical composition comprising another drug; and optionally, instructions for use of both drugs in combination in a method of treatment according to any of items 7 to 20.

It is contemplated that any features described herein can optionally be combined with any of the embodiments of any medical use, pharmaceutical composition, kit, method of treatment, method of manufacturing a medicament and combination therapies of the invention; and any embodiment discussed in this specification can be implemented with respect to any of these.

It will be understood that particular embodiments described herein are shown by way of illustration and not as limitations of the invention.

All publications and patent applications are herein incorporated by reference to the same extent as if each individual publication or patent application was specifically and individually indicated to be incorporated by reference.

The use of the word “a” or “an” may mean “one,” but it is also consistent with the meaning of “one or more,” “at least one,” and “one or more than one”. The use of the term “another” may also refer to one or more. The use of the term “or” in the claims is used to mean “and/or” unless explicitly indicated to refer to alternatives only or the alternatives are mutually exclusive.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. The term “comprises” also encompasses and expressly discloses the terms “consists of” and “consists essentially of”. As used herein, the phrase “consisting essentially of” limits the scope of a claim to the specified materials or steps and those that do not materially affect the basic and novel characteristic(s) of the claimed invention. As used herein, the phrase “consisting of” excludes any element, step, or ingredient not specified in the claim except for, e.g., impurities ordinarily associated with the element or limitation.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “around”, “approximately” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by ±1, 2, 3, 4, 5, 6, 7, 8, 9, 10, 11, 12, 13, 14 or 15%. Accordingly, the term “about” may mean the indicated value ±5% of its value, preferably the indicated value ±2% of its value, most preferably the term “about” means exactly the indicated value (±0%).

The following examples serve to illustrate the present invention and should not be construed as limiting the scope thereof.

### EXAMPLES

### Example 1—Chemical Synthesis and Purification of the Peptidomimetic Compounds

Chemical Synthesis

Materials: Fmoc-protected a-amino acids (include the olefinic amino acids: Fmoc-[(S)-2-(4 pentenyl)alanine]OH, Fmoc-[(R)-2-(4 pentenyl)alanine]OH, Fmoc-[(S)-2-(7 octenyl)alanine]OH, Fmoc-[(R)-2-(7 octenyl)alanine]OH), 2-(6-chloro-1-H-benzotriazole-1-yl)-1,1,3,3-tetramethylaminium hexafluorophosphate (TBTU), resins, dimethylformamide (DMF), N,N-diisopropylethylamine (DIEA), trifluoroacetic acid (TFA), 1,2-dichloroethane (DCE), Grubbs Ru(IV) catalyst and piperidine were purchased from different suppliers.

Briefly, the linear polypeptides were synthesized with automatic synthesizer using Fmoc solid phase peptide chemistry. Only the coupling with olefinic amino acids was performed manually after removing the resins from the reactor vessel.

The ring-closing metathesis reaction was performed in solution with a first generation Grubbs catalyst after cleaving the linear peptide from the resin, as disclosed by Scott J. M. and colleagues (Scott J. M. et al., “Application of Ring-Closing Metathesis to the Synthesis of Rigidified Amino Acids and Peptides”, 1996, J. Am. Chem. Soc., 1996, 118 (40), pp 9606-9614). The deprotected peptide precipitated with methyl-tert-butyl ether at 4° C. and lyophilized.

Purification

The lyophilized peptides were purified by reverse phase HPLC using a C18 column. The peptides were identified by LC-MS-ESI. All the mass spectral data for all the compounds are shown below.

Hplc Conditions:

S09. The compound was purified by HPLC-RP (SepaxGPC-18 column; Pump A: H₂O with 0.1% TFA; Pump B Acetonitrile with 0.1% TFA) using a linear gradient 5%-60% of B in 12 minutes (R. T.=6.55). Purity grade 95.05% by HPLC;

S14. The compound was purified by HPLC-RP (SepaxGPC-18 column; Pump A: H₂O with 0.1% TFA; Pump B Acetonitrile with 0.1% TFA) using a linear gradient 5%-60% of B in 12 minutes (R. T.=7.63). Purity grade 96.67% by HPLC.

Compounds Mass Characterization:

| | | | MW | Mass | Mass |
|---|---|---|---|---|---|
| N. ID | Sequence | | (1H) | (2H) | (3H) |
| S09 | APKXVILKKAXAYILS—OH | calcul. | 1807.3 | 904.7 | 603.4 |
| | | found | 1808.2 | 904.5 | 603.4 |
| S14 | APKXVIFKKAXAYILS—OH | calcul. | 1840.9 | 921.5 | 614.6 |
| | | found | 1842.3 | 921.5 | 614.9 |

### Example 2—Antiproliferative Effects of S09 and S14 in Several Cancer Cell Lines

1. Materials and Methods

Formulation of the Peptidomimetic Compounds

Lyophilised peptidomimetics are dissolved in physiological serum.

Cell Lines

A549, epithelial (lung cancer), ATCC: CCL-185

HL-60, promyeloblast (acute myelocytic leukemia, AML), ECACC: 98070106

MCF-7, epithelial (breast cancer), ECACC: 86012803

MM.1S, B Lymphoblast (multiple myeloma), ATCC: CRL-2974

RAMOS, B lymphocyte (Burkitt's Lymphoma), ATCC: CRL-1596

BJ, fibroblast (normal skin), ATCC® CRL-2522™

Cell Culture

Cell lines A549, MCF-7 and RAMOS were cultured in incubator under CO₂ (6%) at 37° C. in DMEM high glucose (Dulbecco's Modified Eagle Solution, Gibco-BRL 31966-21) medium with 10% fetal bovine serum inactivated (FBS) (Gibco-BRL 10106-169). Cell lines HL-60 and MM.1S were cultured in incubator at 37° C. in RPMI-1640 (Sigma R8758) medium with 10% of fetal bovine serum inactivated (FBS) and 2 mM glutamine (Sigma G7513). Cell lines BJ were cultured in incubator under CO₂ (6%) at 37° C. in Eagle's Minimum Essential Medium (Sigma, M-2279) with 10% fetal bovine serum inactivated (FBS) (Gibco-BRL 10106-169).

During the amplification step and the assays adherent cells were rinsed with DPBS (Dulbecco's Phosphate Buffered Saline, Sigma D1283) three times and afterward treated for 5 minutes with trypsin ([0.5 g/ml]/EDTA [0.2 g/ml]) (Gibco-BRL, 15400054) in solution of DPBS at 37° C., and, once detached, transferred in the culturing medium. No-adherent cells were centrifuged and transferred in the culturing medium. Cells were counted in a Neubauer chamber after labelling with Tripan-Blue. Each assay was performed only when the viability was superior to 90%.

Viability Assay

Cells were seeded at a density of 10000 cells/well in 100 μl of medium in 96 well plates. After 24 h, the compounds to be tested were added to calculate the dose/response curve at the starting concentration of 100 μM with serial dilutions (1:1). Controls are the untreated cells. Each experiment was performed in triplicate.

Cells were incubated with the compounds at the indicated concentrations during 24-72 h in incubator under CO₂ atmosphere at 37° C. Then, cell viability was measured by means of MTT (3-(4,5-dimethylthiazol-2-yl)-2,5-diphenyltetrazolium bromide) assay. Stock solution of MTT (475989 Calbiochem) was 5 mg/ml in PBS. 1×.10 μl/well of MTT solution were added and the plate was incubated for 3-4 hr. The medium was discarded and 100 μl of extracting buffer (PBS 1×, 15% SDS, 50% Na N,N-Dimethylformamide, pH 4.7) were added to each well. Plates were incubated for 16 h at room temperature under orbital shaking. Absorbance at 570 nm was finally measured. As negative control (experiment noise) 3 wells were treated with 20 μl/well of a solution of SDS 10% in H₂O.

Statistics

Data analysis was performed calculating the percentage of cell viability normalized vs. the values of negative control, which was considered equal to 100%. The dose/response curve was fitted through the sigmoidal equation dose-response (variable slope) and the IC₅₀ values were calculated as follow:

Y=Bottom+(Top−Bottom)/(1+10\hat{ }{[(LogIC50−X)*HillSlope]},

where: X is compound concentration (log scale) and Y is the response

Calculations and graphs were conducted using GraphPad Prism (Prism 6 for Windows).

2. Results

The experimental results are summarized in Table 3 below:

TABLE 3
IC50 values in μM :

| | | | Reference compounds | |
|---|---|---|---|---|
| Cell line | S09 | S14 | Int-HI-S6A-F8 | 10058-F4 |
| MM.1S | ≈6.2 | 6.6 ± 0.9 | >100 | >100 |
| HL-60 | 3.5 ± 0.2 | 7.8 ± 0.4 | >100 | 54 ± 2 |
| A549 | 9.4 ± 4.1 | 9.1 ± 0.8 | >100 | >100 |
| Ramos | ≈28 | 55 | >100 | ≈57 |
| MCF7 | 9.0 ± 1 | 3 ± 0.2 | >100 | ≈52 |
| BJ | 47 ± 0.2 | np | np | np |

Reference compounds: Int-HI-S6A-F8 inhibitor, as a positive control (compounds were purchased from Enzo Technology); 10058-F4 as active reference compounds (purchased from Sigma).

As it can be derived from these data, the peptides of formula (I) have shown better specificity and sensitivity than the reference compounds.

### Example 3—Comparative Test Showing that the Particular Position of the Ligand is Critical in the Activity of the Peptides of Formula (I)

In order to demonstrate that the particular position of the ligand (L) in the peptides of formula (I) was critical for the activity, the inventors compared the activity of the peptide S09 with versions thereof differing in the position of the ligand.

TABLE 4
lists the peptides synthesized for
comparative purposes:

| Name | Sequence |
|---|---|
| SEQ ID NO: 5 (IDP-S19) | AXKVVILKXATAYILS |
| SEQ ID NO: 6 (IDP-S21) | APKVXILKKATXYILS |
| SEQ ID NO: 7 (IDP-S22) | APKVVXLKKATAXILS |
| SEQ ID NO: 8 (IDP-S23) | APKVVILKXATAYILXV |
| SEQ ID NO: 9 (IDP-S17) | APKVVIXKKATAYXLS |
| SEQ ID NO: 10 (IDP-S18) | APKVVILXKATAYIXS |

In all cases, the X represents an amino acid of formula:

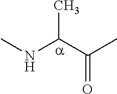

and a L birradical corresponding to —(CH₂)₆—CH═CH—(CH₂)₃— links both Xs through binding to the respective alpha carbon.

The protocol followed for peptide synthesis was substantially the same as the one already disclosed above.

HPLC Conditions:

IDP-S19: The compound was purified by HPLC-RP (C-18 column; Pump A: H₂O with 0.1% TFA; Pump B Acetonitrile with 0.1% TFA) using a linear gradient 47%-57% of B in 20 minutes (R. T.=8.59). Purity grade 95.11% by HPLC;

IDP-S20: The compound was purified by HPLC-RP (C-18 column; Pump A: H₂O with 0.1% TFA; Pump B Acetonitrile with 0.1% TFA) using a linear gradient 53%-63% of B in 20 minutes (R. T.=11.75). Purity grade 97.56% by HPLC;

IDP-S21: The compound was purified by HPLC-RP (C-18 column; Pump A: H₂O with 0.1% TFA; Pump B Acetonitrile with 0.1% TFA) using a linear gradient 35%-45% of B in 20 minutes (R. T.=12.04). Purity grade 95.12% by HPLC;

IDP-S22: The compound was purified by HPLC-RP (C-18 column; Pump A: H₂O with 0.1% TFA; Pump B Acetonitrile with 0.1% TFA) using a linear gradient 40%-50% of B in 20 minutes (R. T.=14.11). Purity grade 95.09% by HPLC;

IDP-S23: The compound was purified by HPLC-RP (C-18 column; Pump A: H₂O with 0.1% TFA; Pump B Acetonitrile with 0.1% TFA) using a linear gradient 50%-60% of B in 20 minutes (R. T.=8.80). Purity grade 98.60% by HPLC;

IDP-S17: The compound was purified by HPLC-RP (C-18 column; Pump A: H₂O with 0.1% TFA; Pump B Acetonitrile with 0.1% TFA) using a linear gradient 5%-60% of B in 12 minutes (R. T.=7.01). Purity grade 99.11% by HPLC; and

IDP-S18: The compound was purified by HPLC-RP (C-18 column; Pump A: H₂O with 0.1% TFA; Pump B Acetonitrile with 0.1% TFA) using a linear gradient 5%-60% of B in 12 minutes (R. T.=8.2). Purity grade 97.8% by HPLC.

TABLE 5
mass characterization

| | | | MW (Found, |
|---|---|---|---|
| Name | Sequence | MW (calc) | 1H) |
| IDP-S19 | AXKVVILKXATAYILS | 1781.01 | 1782.05 |
| IDP-S21 | APKVXILKKATXYILS | 1836.09 | 1837.1 |
| IDP-S22 | APKVVXLKKATAXILS | 1729.97 | 1730.97 |
| IDP-S23 | APKVVILKXATAYILXV | 1890.18 | 1891.3 |
| IDP-S17 | APKVVIXKKATAYXLS | 1780.2 | 1781.2 |
| IDP-S18 | APKVVILXKATAYIXS | 1765.2 | 1766.6 |

Following the same protocol as the one disclosed in previous sections, the following activity data were obtained:

TABLE 6

| | | Compound (IC50, uM) | | | | | |
|---|---|---|---|---|---|---|---|
| Cell line | IDP-S09 | IDP-S19 | IDP-S21 | IDP-S22 | IDP-S23 | IDP-S17 | IDP-S18 |
| MM.1S | ≈6.2 | >100 | >100 | >100 | >100 | >100 | >100 |
| HL-60 | 3.5 ± 0.2 | >100 | >100 | >100 | >100 | >100 | >100 |
| A549 | 9.4 ± 4.1 | >100 | >100 | >100 | >100 | >100 | >100 |
| Ramos | ≈28 | >100 | >100 | >100 | >100 | >100 | >100 |
| MCF7 | 9.0 ± 1 | >100 | >100 | >100 | >100 | >100 | >100 |

The wild-type sequence SEQ ID NO: 11 (Pro-Lys-Val-Val-11e-Leu-Lys-Lys-Ala-Thr-Ala-Tyr-Ile) does not present growth inhibition activity. Surprisingly, when a ligand as described herein is linking positions 3^{rd} and 10^{th} of the wild type sequence [corresponding to positions X1 and X3 of a compound of formula (I)] antiproliferative properties in the uM range are observed. Conversely, the other versions (where a bridge was created between other positions in the peptidic sequence) did not confer the compounds with significant activity. This is something surprising because, up to date, cross-linked (stapled) peptides have been disclosed as presenting improved stability with respect to the original peptide sequence. There is no hint however in the prior art about the possible effect of a stapler in the activation of the wild-type peptide sequence which, originally, was inactive.

### Example 4—Efficacy of S09 in Drug Resistant Multiple Myeloma Cell Lines

1. Materials and Methods

Formulation of the Peptidomimetic Compounds

Lyophilised peptidomimetics are dissolved in physiological serum.

Cell Lines:

NCI-H929, lymphoblast (myeloma), ATCC® CRL-9068™

OPM-2, lymphoblast (myeloma), DSMZ No. ACC50

MM144, lymphoblast (myeloma), University of Salamanca

MM1R, lymphoblast, (myeloma), ATCC® CRL-2975™, resistant to Dexamethasone

RPMI-8266, lymphoblast (myeloma), ATCC® CCL155™

RPMI-8266-LR5, lymphoblast (myeloma), selected for resistance to Melphalan (Bellamy W T et al., Cancer Res. 1991 Feb. 1; 51(3):995-1002).

U266, lymphoblast (myeloma), ATCC® TIB196™

U266DOX4, lymphoblast (myeloma), selected for resistance to Doxorubicin (Alvarez-Fernández et al. Clin Cancer Res. 2013 May 15; 19(10):2677-87).

U266-LR7, lymphoblast (myeloma), selected for resistance to Melphalan (Alvarez-Fernández et al. Clin Cancer Res. 2013 May 15; 19(10):2677-87).

Cell Culture

All cell lines were cultured in incubator at 37° C. in RPMI-1640 (Sigma R8758) medium with 10% of fetal bovine serum inactivated (FBS) and 2 mM glutamine (Sigma G7513). During the amplification step cells were centrifuged and transferred into the culturing medium. Cells were counted in a Neubauer chamber after labelling with Tripan-Blue. Each assay was performed only when the viability was superior to 90%.

Viability Assay

Cells were seeded at a density of 10000 cells/well in 100 μl of medium in 96 well plates. After 24 h, the compounds to be tested were added to calculate the dose/response curve at the starting concentration of 100 μM with serial dilutions (1:1). Controls are the untreated cells. Each experiment was performed in triplicate.

Cells were incubated with the peptidomimetic compound (S09) at the indicated concentrations during 24-72 h in incubator under CO₂ atmosphere at 37° C. Then, cell viability was measured by means of MTT (3-(4,5-dimethylthiazol-2-yl)-2,5-diphenyltetrazolium bromide) assay. Stock solution of MTT (475989 Calbiochem) was 5 mg/ml in PBS. 1×.10 μl/well of MTT solution were added and the plate was incubated for 3-4 hr. The medium was discarded and 100 μl of extracting buffer (PBS 1×, 15% SDS, 50% Na N,N-Dimethylformamide, pH 4.7) were added to each well. Plates were incubated for 16 h at room temperature under orbital shaking. Absorbance at 570 nm was finally measured. As negative control (experiment noise) 3 wells were treated with 20 μl/well of a solution of SDS 10% in H₂O.

Statistics

Data analysis was performed calculating the percentage of cell viability normalized vs. the values of negative control, which was considered equal to 100%. The dose/response curve was fitted through the sigmoidal equation dose-response (variable slope) and the IC₅₀ values were calculated as follow:

Y=Bottom+(Top−Bottom)/(1+10\hat{ }{[(LogIC50−X)*HillSlope]},

where: X is compound concentration (log scale) and Y is the response Calculations and graphs were conducted using GraphPad Prism (Prism 6 for Windows).

2. Results

The objective of this study was to determine the in vitro anti-proliferative properties of S09 in various multiple myeloma (MM) cell lines, some of these cell lines having been described to be resistant to drugs generally used in the primary treatment of MM. The obtained IC50 (μM) values, which correspond to the concentration of an agent that causes a 50% growth inhibition, are summarized in Table 7 below:

TABLE 7
IC50 values in μM for S09

| Cell line | S09 | |
|---|---|---|
| H929 | 3.6 | μM |
| OPM2 | 4.8 | μM |
| MM144 | 6.8 | μM |
| MM1R | 8.3 | μM |
| MM1S | 7.5 | μM |
| RPMI | 7.4 | μM |
| RPMI-LR5 | 11.4 | μM |
| U266 | 7.5 | μM |
| U266DOX4 | 5 | μM |
| U266LR7 | 9.7 | μM |

S09 shows high anti-cancer activity in several MM cell lines. In particular, it shows efficacy almost in the same range in different myeloma cells resistant to approved MM treatment, such as Melphalan (RPMI-LR5 and U266-LR7), Doxorubicine (U266Dox4) and Dexamethasone (MM1R).

### Example 5—Determination of In Vitro Synergy for S09 Drug Combinations

1. Materials and Methods

Cell Line

MM1S, lymphoblast (myeloma), ATCC® CRL-2974™

Cell Culture

MM1S cells were cultured in incubator at 37° C. in RPMI-1640 (Sigma R8758) medium with 10% of fetal bovine serum inactivated (FBS) and 2 mM glutamine (Sigma G7513).

During the amplification step cells were centrifuged and transferred in the culturing medium. Cells were counted in a Neubauer chamber after labelling with Tripan-Blue. Each assay was performed only when the viability was superior to 90%.

In Vitro Synergy

MM1S cells were treated for 24 h with different doses of S09, dexamethasone, bortezomib and cyclophosphamide in monotherapy and in double and triple combinations. Different dose combinations were explored for each triple combination keeping a constant ratio between them. The following concentrations were used in the triple combinations (corresponding concentrations were used in monotherapy and in double combinations tested):
dexamethasone:bortezomib (5 nM:1 nM and 5 nM:2 nM), with serial concentrations of S09 of 0.3 uM; 0.6 uM; 1.25 uM and 2.5 uM; and
dexamethasone:cyclophosphamide (5 nM:2.5 uM and 5 nM:5 uM), with serial concentrations of S09 of 0.3 uM; 0.6 uM; 1.25 uM and 2.5 uM.

The viability was measured by means of MTT (3-(4,5-dimethylthiazol-2-yl)-2,5-diphenyltetrazolium bromide) assay (as defined before).

The potency of the combination was quantitated with the Calcusyn software (Biosoft, Ferguson, Mo., USA), which is based on the Chou Talalay method (Chou T C, Talalay P. Adv Enzyme Regul. 1984; 22:27-55) that calculates a combination index (CI) with the following interpretation: Cl >1: antagonistic effect, Cl=1: additive effect and Cl<1 synergistic effect.

2. Results

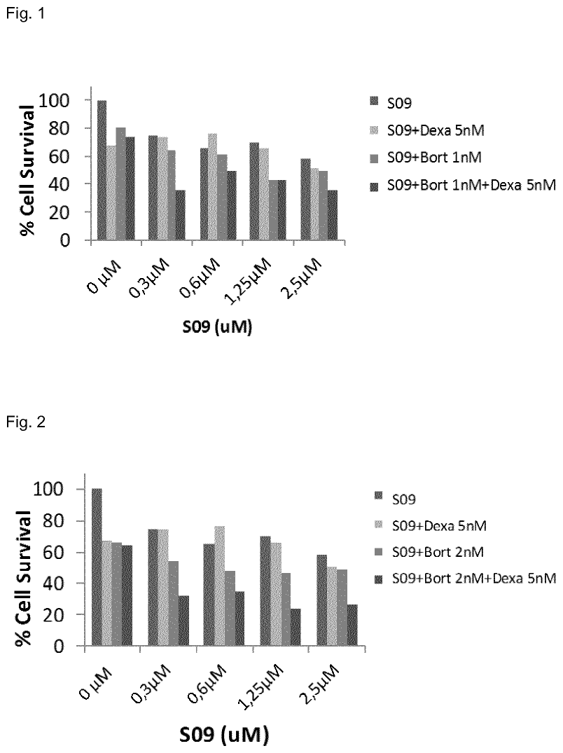

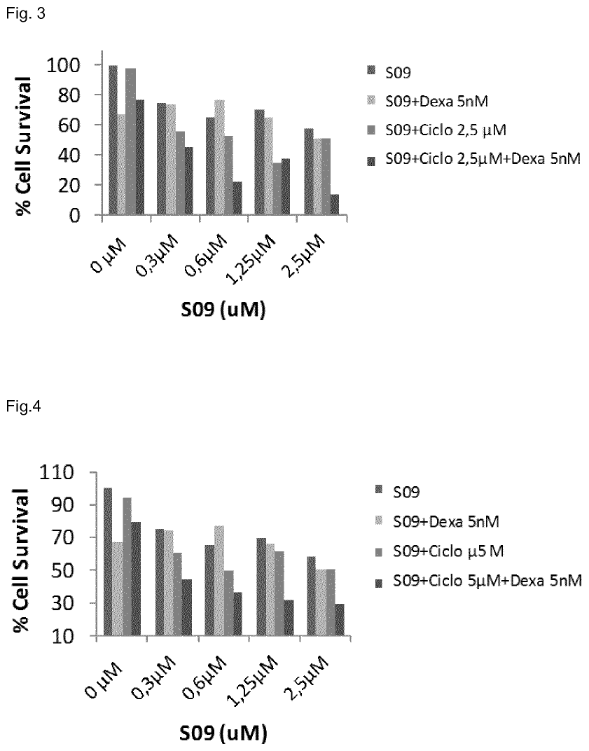

The objective of this study was to determine the ability of S09 to potentiate in vitro the antitumor activity of other anti-myeloma agents. The experimental results are illustrated in FIGS. 1, 2, 3 and 4.

The calculated synergy index (CI) for the double and triple combinations obtained with regards to the mono- and double therapies, respectively, is provided in the tables below, for each of the S09 tested concentrations (i.e., 0.3, 0.6, 1.2 and 2.5 uM).

TABLE 8
S09, bortezomib and dexamethasone

| Conc S09 | DC Bort | DC Bort | DC Dexa | TC Bort | TC Bort |
|---|---|---|---|---|---|
| (uM) | 1 nM | 2 nM | 5 nM | 1 nM&Dexa5 nM | 2 nM&Dexa5 nM |
| 0.3 | 0.422 | 0.68 | 0.699 | 0.274 | 0.476 |
| 0.6 | 0.414 | 0.612 | 1.13 | 0.403 | 0.503 |
| 1.25 | 0.312 | 0.668 | 1.36 | 0.375 | 0.389 |
| 2.5 | 0.526 | 0.821 | 0.379 | 0.309 | 0.001 |

TABLE 9
S09, cyclophosphamide and dexamethasone

| Cone S09 | DC Cyclo | DC Cyclo | DC Dexa | TC Cyclo | TC Cyclo |
|---|---|---|---|---|---|
| (uM) | 2.5 uM | 5 uM | 5 nM | 2.5 uM&Dexa5 nM | 5 uM&Dexa5 nM |
| 0.3 | 0.163 | 0.473 | 0.699 | 0.251 | 0.416 |
| 0.6 | 0.164 | 0.39 | 1.13 | 0.131 | 0.351 |
| 1.25 | 0.218 | 1.01 | 1.36 | 0.22 | 0.323 |
| 2.5 | 0.177 | 0.657 | 0.379 | 0.1 | 0.308 |

S09 showed good synergy with standard of care drugs used in multiple myeloma treatment, namely bortezomib, cyclophosphamide and dexamethasone, as it can be observed by means of the MM1S cells survival results after 24 hr incubation with the drug treatments both in double and triple combinations (as shown by a Cl lower than 1), specially with bortezomib and cyclophosphamide 2.5 uM.

Moreover, the triple combination treatments with S09, namely:
i. S09, dexamethasone and bortezomib; and
ii. S09, dexamethasone and cyclophosphamide;

were shown to provide a very good synergy.

Most synergistic combinations were shown to be:
S092.5 uM, bortezomib 2 nM and dexamethasone 5 nM; and
S092.5 uM, cyclophosphamide 2.5 uM and dexamethasone 5 nM.

### Example 6—Determination of In Vivo Synergy for S09 Drug Combinations

1. Materials and Methods

Animals

CB17-SCID immunosuppressed mice (female) of 6-7 weeks old housed and handled in a pathogen-free zone. They were purchased from Janvier Labs (France). All experiments were carried out within the facilities of the University of Salamanca (Spain).

Groups Studied

Control: Medium (PBS), intraperitoneally (i.p.), each 12 hours on days 1 to 5.

Treatment Groups:
Monotherapy:
   Bortezomib 0.5 mg/kg i.p. on days 1 and 4.
   Cyclophosphamide 50 mg/kg i.p. on days 1 and 4
   Dexamethasone 0.5 mg/kg i.p. on days 1 and 4
   S09 4 mg/kg i.p. each 12 hours on days 1 to 7
Double combinations:
   Bortezomib 0.5 mg/kg i.p. on days 1 and 4+S09 4 mg/kg i.p. each 12 hours on days 1 to 7
   Cyclophosphamide 50 mg/kg i.p. on days 1 and 4+S09 4 mg/kg i.p. each 12 hours on days 1 to 7
   Dexamethasone 0.5 mg/kg i.p. on days 1 and 4+S09 4 mg/kg i.p. each 12 hours on days 1 to 7
   Bortezomib 0.5 mg/kg i.p. on days 1 and 4+Cyclophosphamide 50 mg/kg i.p. on days 1 and 4
   Cyclophosphamide 50 mg/kg i.p. on days 1 and 4+Dexamethasone 0.5 mg/kg i.p. on days 1 and 4
   Dexamethasone 0.5 mg/kg i.p. on days 1 and 4+Bortezomib 0.5 mg/kg i.p. on days 1 and 4
Triple combinations:
   Bortezomib 0.5 mg/kg i.p. on days 1 and 4+Dexamethasone 0.5 mg/kg i.p. on days 1 and 4+S09 4 mg/kg i.p. each 12 hours on days 1 to 7
   Cyclophosphamide 50 mg/kg i.p. on days 1 and 4+Dexamethasone 0.5 mg/kg i.p. on days 1 and 4+S09 4 mg/kg i.p. each 12 hours on days 1 to 7

Subcutaneous Xenograft Model

Mice were shaved on the right flank and anesthetized by inhalation to decrease their mobility, were inoculated subcutaneously with 3×106 MM1S cells in 50 μl of RPMI-1640 medium and 50 μl of Corning @ Matrigel® Basement Membrane Matrix. When the tumors became palpable (at 30 days), the mice were randomized into the different groups (5 mice in the control group and 4 in the remaining groups) according to the tumor volume (mm³). This was estimated by measurements with a caliper of the two tumor diameters and using the following formula of a spheroid:

V=(a·b\hat{ }2·π)/6

Where a and b correspond to the longest and shortest diameter, respectively. Tumor volume was monitored three times per week.

The treatment endpoint was determined by the value of tumor volume in the mouse (a range of 2000-2200 mm3).

2. Results

The objective of this study was to determine the ability of S09 to potentiate in vivo the antitumor activity of other agents used in the treatment of multiple myeloma. The following agents were evaluated in combination with S09: bortezomib (B), cyclophosphamide (C) and dexamethasone (D) in double and triple combinations.

The experimental data are summarized in Table 10 below, where the tumor volume and normalized tumor volume % are reported for day of treatment 1, 5, 10, 15 and 24 of the groups of untreated mice (control), treated with one drug (monotherapy), treated with a cocktail of 2 drugs (double combination) and treated with a cocktail of three drugs (triple combination).

TABLE 10
Tumor volume and normalized tumor volume percentage (%) measured
at different days of treatment for all the groups

| | | | Day of treatment | | | | |
|---|---|---|---|---|---|---|---|
| | | | 1 | 5 | 10 | 15 | 24 |
| Volume | Untreated | Vehicle | 120.09 | 191.62 | 420.05 | 998.63 | 1978.66 |
| | Monotherapy | B | 119.96 | 165.98 | 276.48 | 564.56 | 1691.81 |
| | | C | 120.75 | 189.48 | 298.83 | 667.23 | 1512.04 |
| | | D | 120.35 | 133.13 | 325.76 | 631.72 | 1451.67 |
| | | 9 | 119.55 | 178.12 | 321.49 | 564.86 | 1370.87 |
| | Double | BD | 119.82 | 148.86 | 290.73 | 561.28 | 1397.88 |
| | combination | B9 | 119.71 | 101.07 | 160.01 | 170.58 | 485.86 |
| | | CD | 120.19 | 160.63 | 308.46 | 631.43 | 1332.71 |
| | | C9 | 119.91 | 117.06 | 178.32 | 226.41 | 552.68 |
| | | D9 | 119.23 | 138.38 | 303.89 | 330.74 | 823.99 |
| | Triple | BD9 | 119.24 | 92.69 | 187.39 | 123.96 | 280.62 |
| | combination | CD9 | 119.99 | 121.17 | 213.75 | 283.17 | 573.91 |
| Volume | Untreated | Vehicle | 100.00 | 159.56 | 349.78 | 831.57 | 1647.65 |
| % | Monotherapy | B | 100.00 | 138.36 | 230.48 | 470.62 | 1410.31 |
| | | C | 100.00 | 156.92 | 247.48 | 552.57 | 1252.21 |
| | | D | 100.00 | 110.62 | 270.68 | 524.90 | 1206.21 |
| | | 9 | 100.00 | 148.99 | 268.92 | 472.49 | 1146.69 |
| | Double | BD | 100.00 | 124.24 | 242.64 | 468.44 | 1166.65 |
| | combination | B9 | 100.00 | 84.43 | 133.66 | 142.49 | 405.86 |
| | | CD | 100.00 | 133.65 | 256.64 | 525.36 | 1108.84 |
| | | C9 | 100.00 | 97.62 | 148.71 | 188.82 | 460.91 |
| | | D9 | 100.00 | 116.06 | 254.88 | 277.40 | 691.09 |
| | Triple | BD9 | 100.00 | 77.73 | 157.15 | 103.96 | 235.34 |
| | combination | CD9 | 100.00 | 100.98 | 178.14 | 235.99 | 478.30 |

S09 showed very good synergy in vivo with drugs that are generally present in multiple myeloma treatment regimens, as it can be observed by means of tumor growth reduction in the mice xenograft model. At day 24, when the tumor volume of the untreated mice (control) reaches the maximum value (1978.66 mm³) the efficacy reported for bortezomib, cyclophosphamide and dexamethasone in monotherapy is improved when combined with S09 (i.e. double combinations), showing a reduction of tumor growth of 76%, 72% and 58%, respectively, with respect to the untreated control.

The triple combination of cyclophosphamide, dexamethasone and S09 showed a tumor growth reduction of 71% and the triple combination of dexamethasone, bortezomib and S09 reached a reduction of tumor volume of almost 90% (86%) with respect to the untreated control.

### Example 7—Chemical Synthesis and Purification of the IDP-P1708160, and IDP-P1708161 Peptidomimetic Compounds

Chemical Synthesis

Materials: Fmoc-protected a-amino acids, 2-(6-chloro-1-H-benzotriazole-1-yl)-1,1,3,3-tetramethylaminium hexafluorophosphate (TBTU), resins, dimethylformamide (DMF), N,N-diisopropylethylamine (DIEA), trifluoroacetic acid (TFA), 1,2-dichloroethane (DCE), tris(2-carboxyethyl)phosphine (TCEP), 1-8-di-bromide-octane, 1,2-bis(2-bromo-ethoxy)ethane, 4((4-bromomethyl)phenyl)benzylbromide and piperidine were purchased from different suppliers.

Briefly, the linear polypeptides were synthesized with automatic synthesizer using Fmoc solid phase peptide chemistry. After selective deprotection of cysteine side chains, coupling reactions with 1-8-di-bromide-octane, 4((4-bromomethyl)phenyl)benzylbromide to obtain IDP-P1708160 and IDP-P1708161, respectively were conducted in DMF, in the presence of TCEP at room temperature for 2 hr (Doron C. Greenbaum et al. “Development of alpha-Helical Calpain Probes by Mimicking a Natural Protein-Protein Interaction”, JACS 2012). The peptide was cleaved and side chains protecting groups removed with TFA in DCM. The deprotected peptide precipitated with methyl-tert-butyl ether at 4° C. and lyophilized.

Purification

The lyophilized peptides were purified by reverse phase HPLC using a C18 column. The peptides were identified by LC-MS-ESI. All the mass spectral data for all the compounds are shown below.

HPLC Conditions:

IDP-P1708160. The compound was purified by HPLC-RP (SepaxGPC-18 column; Pump A: H₂O with 0.1% TFA; Pump B Acetonitrile 80% with 0.1% TFA) using a linear gradient 41%-61% of B in 20 minutes (R. T.=9.95). Purity grade 97.71% by HPLC;

IDP-P1708161. The compound was purified by HPLC-RP (SepaxGPC-18 column; Pump A: H₂O with 0.1% TFA; Pump B Acetonitrile 80% with 0.1% TFA) using a linear gradient 40%-60% of B in 20 minutes (R. T.=10.26). Purity grade 95.13% by HPLC.

TABLE 11
Compounds mass characterization:

| N. ID | | MW(1H) | Mass (2H) | Mass (3H) |
|---|---|---|---|---|
| P1708160 | calcul. | 1831.38 | (−) 914.7 | |
| | found | | (−) 914.7 | |
| P1708161 | calcul. | 1898.06 | (+) 950.0 | (+)633.7 |
| | found | | (+) 950.5 | (+) 634.1 |

### Example 8—Antiproliferative Effects of IDP-P1708160, and IDP-P1708161 in Several Cancer Cell Lines

1. Materials and Methods

Formulation of the Peptidomimetic Compounds

Lyophilised peptidomimetics are dissolved in physiological serum.

Cell Lines

A549, epithelial (lung cancer), ATCC: CCL-185

MBA-MD231, epithelial (breast cancer), ECACC: 86012803

MM.1S, B Lymphoblast (multiple myeloma), ATCC: CRL-2974

NCI-H128, epithelial (small cell lung cancer), ATCC: HTB-120™

Cell Culture

Cell lines A549 and MBA-MD231 were cultured in incubator under CO₂ (6%) at 37° C. in DMEM high glucose (Dulbecco's Modified Eagle Solution, Gibco-BRL 31966-21) medium with 10% fetal bovine serum inactivated (FBS) (Gibco-BRL 10106-169). Cell lines NCI-H128 and MM.1S were cultured in incubator at 37° C. in RPMI-1640 (Sigma R8758) medium with 10% of fetal bovine serum inactivated (FBS) and 2 mM glutamine (Sigma G7513).

During the amplification step and the assays adherent cells were rinsed with DPBS (Dulbecco's Phosphate Buffered Saline, Sigma D1283) three times and afterward treated for 5 minutes with trypsin ([0.5 g/ml]/EDTA [0.2 g/ml]) (Gibco-BRL, 15400054) in solution of DPBS at 37° C., and, once detached, transferred in the culturing medium. No-adherent cells were centrifuged and transferred in the culturing medium. Cells were counted in a Neubauer chamber after labelling with Tripan-Blue. Each assay was performed only when the viability was superior to 90%.

Viability Assay

Cells were seeded at a density of 10,000 cells/well in 100 μl of medium in 96 well plates. After 24 h, the compounds to be tested were added to calculate the dose/response curve at the starting concentration of 100 μM with serial dilutions (1:1). Controls are the untreated cells. Each experiment was performed in triplicate.

Cells were incubated with the compounds at the indicated concentrations during 24-72 h in incubator under CO₂ atmosphere at 37° C. Then, cell viability was measured by means of MTT (3-(4,5-dimethylthiazol-2-yl)-2,5-diphenyltetrazolium bromide) assay. Stock solution of MTT (475989 Calbiochem) was 5 mg/ml in PBS. 1×.10 μl/well of MTT solution were added and the plate was incubated for 3-4 hr. The medium was discarded and 100 μl of extracting buffer (PBS 1×, 15% SDS, 50% Na N,N-Dimethylformamide, pH 4.7) were added to each well. Plates were incubated for 16 h at room temperature under orbital shaking. Absorbance at 570 nm was finally measured. As negative control (experiment noise) 3 wells were treated with 20 μl/well of a solution of SDS 10% in H₂O.

Statistics

Data analysis was performed calculating the percentage of cell viability normalized vs. the values of negative control, which was considered equal to 100%. The dose/response curve was fitted through the sigmoidal equation dose-response (variable slope) and the IC₅₀ values were calculated as follow:

Y=Bottom+(Top−Bottom)/(1+10\hat{ }{[(LogIC50−X)*HillSlope]},

where: X is compound concentration (log scale) and Y is the response Calculations and graphs were conducted using GraphPad Prism (Prism 6 for Windows).

2. Results

The experimental results are summarized in the table below:

TABLE 12
IC50 values in μM:

| Cell line | IDP-P1708160 | IDP-P1708161 | S09 |
|---|---|---|---|
| MM.1S | 48 | 33 ± 15 | 6.7 ± 1 |
| A549 | 65 ± 17 | 42 ± 6 | 6.3 ± 0.5 |
| MBA-MD231 | 42 ± 2 | 29 ± 2 | 4.9 ± 0.3 |
| NCI-H128 | 16 ± 1 | 14 ± 1 | 3 ± 1 |

As it can be derived from these data, peptides of formula (I) with ligands (L) of formula (IIIa) other than the one used in the S09 and S14 compounds, are shown to provide good cytotoxic activity, comparable to that of S09, in various cancer cell lines.

### Example 9—Efficacy of IDP-P1708160, IDP-P1708161, and S14 in Drug Resistant Multiple Myeloma Cell Lines

1. Materials and Methods

Formulation of the Peptidomimetic Compounds

Lyophilised peptidomimetics are dissolved in physiological serum.

Cell Lines:

MM1R, lymphoblast, (myeloma), ATCC® CRL-2975™, resistant to Dexamethasone

RPMI-8266, lymphoblast (myeloma), ATCC® CCL155™

RPMI-8266-LR5, lymphoblast (myeloma), selected for resistance to Melphalan (Bellamy W T et al., Cancer Res. 1991 Feb. 1; 51(3):995-1002).

U266DOX4, lymphoblast (myeloma), selected for resistance to Doxorubicin (Alvarez-Fernandez et al. Clin Cancer Res. 2013 May 15; 19(10):2677-87).

U266-LR7, lymphoblast (myeloma), selected for resistance to Melphalan (Alvarez-Fernandez et al. Clin Cancer Res. 2013 May 15; 19(10):2677-87).

Cell Culture

All cell lines were cultured in incubator at 37° C. in RPMI-1640 (Sigma R8758) medium with 10% of fetal bovine serum inactivated (FBS) and 2 mM glutamine (Sigma G7513).

During the amplification step cells were centrifuged and transferred into the culturing medium. Cells were counted in a Neubauer chamber after labelling with Tripan-Blue. Each assay was performed only when the viability was superior to 90%.

Viability Assay

Cells were seeded at a density of 10,000 cells/well in 100 μl of medium in 96 well plates. After 24 h, the compounds to be tested were added to calculate the dose/response curve at the starting concentration of 40 μM with serial dilutions (1:1). Controls are the untreated cells. Each experiment was performed in triplicate.

Cells were incubated with the peptidomimetic compounds at the indicated concentrations during 24-72 h in incubator under CO₂ atmosphere at 37° C. Then, cell viability was measured by means of MTT (3-(4,5-dimethylthiazol-2-yl)-2,5-diphenyltetrazolium bromide) assay. Stock solution of MTT (475989 Calbiochem) was 5 mg/ml in PBS. 1×.10 μl/well of MTT solution were added and the plate was incubated for 3-4 hr. The medium was discarded and 100 μl of extracting buffer (PBS 1×, 15% SDS, 50% Na N,N-Dimethylformamide, pH 4.7) were added to each well. Plates were incubated for 16 h at room temperature under orbital shaking. Absorbance at 570 nm was finally measured. As negative control (experiment noise) 3 wells were treated with 20 μl/well of a solution of SDS 10% in H₂O.

Statistics

Data analysis was performed calculating the percentage of cell viability normalized vs. the values of negative control, which was considered equal to 100%. The dose/response curve was fitted through the sigmoidal equation dose-response (variable slope) and the IC₅₀ values were calculated as follow:

Y=Bottom+(Top−Bottom)/(1+10\hat{ }{[(LogIC50−X)*HillSlope]},

where: X is compound concentration (log scale) and Y is the response

Calculations and graphs were conducted using GraphPad Prism (Prism 6 for Windows).

2. RESULTS

The objective of this study was to determine the in vitro anti-proliferative properties of IDP-P1708160, IDP-P1708161 in various multiple myeloma (MM) cell lines, some of these cell lines having been described to be resistant to drugs generally used in the primary treatment of MM. The obtained IC50 (μM) values, which correspond to the concentration of an agent that causes a 50% growth inhibition, are summarized in the tables below:

TABLE 13
IC50 after 24 hr values in μM for

| Cell line | P1708160 | P1708161 | | S14 |
|---|---|---|---|---|
| MM1R | 15 μM | 12.5 | μM | 15 μM |
| MM1S | 35 μM | 10 | μM | 7 μM |
| RPMI | 15 μM | 5 | μM | 10 μM |
| RPMI-LR5 | 35 μM | 35 | μM | 10 μM |
| U266DOX4 | 10 μM | 10 | μM | 15 μM |
| U266LR7 | 2.5 μM | 5 | μM | 10 μM |

TABLE 14
IC50 after 72 hr values in μM for

| | | | |
|---|---|---|---|
| Cell line | P1708160 | P1708161 | S14 |

| | | | | | |
|---|---|---|---|---|---|
| MM1R | 10 | μM | 15 | μM | 15 μM |
| MM1S | 5 | μM | 15 | μM | 7 μM |
| RPMI | 15 | μM | 7.5 | μM | 10 μM |
| RPMI-LR5 | 7.5 | μM | 35 | μM | 10 μM |
| U266DOX4 | 7.5 | μM | 5 | μM | 15 μM |
| U266LR7 | 2.5 | μM | 5 | μM | 10 μM |

Accordingly, IDP-P1708160, IDP-P1708161 and S14 also showed good anti-cancer activity in several MM cell lines resistant to standard treatments, namely, against cell lines resistant to Melphalan (RPMI-LR5 and U266-LR7), Doxorubicine (U266Dox4) and Dexamethasone (MM1R).

1. A compound of formula (I): 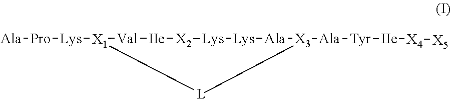
wherein X₂ is a non-polar amino acid, preferably selected from the group consisting of Leu and Phe; and
wherein X₄ is an amino acid, preferably Leu;
wherein X₅ is an amino acid, preferably Ser;
wherein X₁ and X₃ are independently selected and have formula (II): 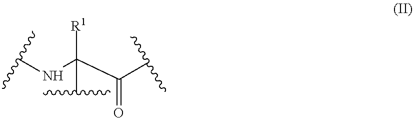
wherein R₁ is H or a monoradical selected from the group consisting of: (C₁-C₁₀)alkyl, (C₂-C₁₀)alkenyl, (C₂-C₁₀)alkynyl, (C₁-C₁₀)alkyl-O—(C₁-C₁₀)alkyl, (C₁-C₁₀)alkyl-C(═O)—(C₁-C₁₀)alkyl, (C₁-C₁₀)alkyl-O—C(O)—(C₁-C₁₀)alkyl, (C₁-C₁₀)alkyl-C(O)—NR₂—(C₁-C₁₀)alkyl, (C₁-C₁₀)alkyl-S—(C₁-C₁₀)alkyl, (C₁-C₁₀)alkyl-SR₃—(C₁-C₁₀)alkyl, (C₁-C₁₀)alkyl-S(═O)₂—(C₁-C₁₀)alkyl, (C₁-C₁₀)alkyl-S(═O)—(C₁-C₁₀)alkyl, (C₁-C₁₀)alkyl-O—S(═O)₂—O—(C₁-C₁₀)alkyl, (C₁-C₁₀)alkyl-NR₄—(C₁-C₁₀)alkyl; and
a ring system comprising from 3 to 14 carbon atoms, the system comprising from 1 to 3 rings, where:
each one of the rings is saturated, partially unsaturated, or aromatic;
the rings are isolated, partially or totally fused,
each one of the members forming the ring system is selected from the group consisting of: —CH—, —CH₂—, —NH—, —N—, —SH—, —S—, and —O—; and
the ring system is optionally substituted by one or more radicals independently selected from the group consisting of halogen, —OH, —NO₂, (C₁-C₁₀)alkyl, (C₁-C₁₀)haloalkyl, and (C₁-C₁₀)alkyl-O—; and
R₂, R₃ and R₄ are monoradicals independently selected from the group consisting of: hydrogen, (C₁-C₁₀)alkyl, (C₂-C₁₀)alkenyl, and (C₂-C₁₀)alkynyl; and
wherein L is a biradical bound to X₁ and X₃ via the alpha carbon, and is selected from the group consisting of: —O—, O—(C₁-C₁₀)alkyl-O—, O—(C₁-C₁₀)alkenyl-O—, C(═O), C(═O)NR₅, C(═O)O, NR₆, S—S—, S—(C₁-C₁₀)alkyl-S, S—(C₁-C₁₀)alkenyl-S— and a ring system consisting of one ring from 3 to 6 members, the ring:
being saturated, partially unsaturated, or aromatic;
each one of the members forming the ring system being selected from the group consisting of: —CH—, —CH₂—, —NH—, —N—, —SH—, —S—, and —O—; and
the ring system being optionally substituted by one or more radicals independently selected from the group consisting of halogen, —OH, —NO₂, (C₁-C₁₀)alkyl, (C₁-C₁₀)haloalkyl, and (C₁-C₁₀)alkyl-O—; and
R₅ and R₆ are radicals independently selected from the group consisting of: —H and (C₁-C₁₀)alkyl (C₁-C₁₀)alkyl, (C₂-C₁₀)alkenyl, and (C₂-C₁₀)alkynyl;
for use in a method of treating a subject, preferably a human, having multiple myeloma.

2. The compound of formula (I) for use in a method of treatment according to claim 1, wherein X₂ is selected from the group consisting of Leu and Phe; preferably X₁ and X₃ are selected from Ala and Gly, and wherein L is of formula (IIIa):
—(CH₂)_{y}P—(CH₂)ₐ-(Q)_{b}-(CH₂)_{c}—(V)_{d}—(CH₂)_{z}— (IIIa)
wherein
P, Q and V are each independently selected from the group comprising O, S, NH, CONH, C(O)O, C₁-C₆ alkylene, arylene groups such as phenylene and heteroarylene groups such as triazolene, optionally substituted by an halogen;
y and z are integer values each independently selected from 1 to 10;
a and c are integer values each independently selected from 0 to 10; and
b and d are integer values each independently selected from 0 to 3.

3. The compound of formula (I) for use in a method of treatment according to claim any of claim 1 or 2, wherein X₂ is selected from the group consisting of Leu and Phe; and wherein L is of formula (IIIb):
—(CH₂)_{y}—CH═CH—(CH₂)_{z}— (IIIb)
wherein y and z are the same or different and are integer values selected from 1 to 10, preferably are independently selected from 3 to 6, more preferably are independently selected from 3 and 6.

4. The compound of formula (I) for use in a method of treatment according to any of claims 1 to 3, wherein said compound is selected from the group consisting of S09 (SEQ ID NO: 3), S14 (SEQ ID NO: 4), and combinations thereof.

5. The compound of formula (I) for use in a method of treatment according to any of claim 1 or 2, wherein X₂ is selected from the group consisting of Leu and Phe; and wherein L is of formula (IIIc): 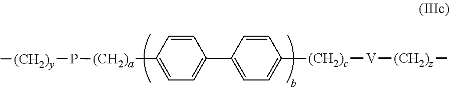
preferably, wherein, P and V are each independently selected from the group consisting of O and S; y, and z are 1; a is selected from 1 or from 6 to 10; b is 0 or 1; and c is 0 or 1.

6. The compound of formula (I) for use in a method of treatment according to any of claims 1-2 or 5, wherein said compound is selected from the group consisting of IDP-P1708160 (SEQ ID NO: 13), IDP-P1708161 (SEQ ID NO: 14), and combinations thereof.

7. The compound of formula (I) for use in a method of treatment according to any of claims 1 to 6, wherein said multiple myeloma is resistant, refractory or relapsing multiple myeloma, preferably wherein said multiple myeloma is resistant, refractory or relapsing to a previous treatment.

8. The compound of formula (I) for use in a method of treatment according to any of claims 1 to 7, wherein said treatment comprises the administration of a compound of formula (I) in combination with another drug.

9. The compound of formula (I) for use in a method of treatment according to claim 8, wherein said other drug is selected from the group consisting of an alkylating agent, a corticosteroid, a proteasome inhibitor, and combinations thereof.

10. The compound of formula (I) for use in a method of treatment according to claim 9, wherein said treatment comprises the administration of a compound of formula (I), a corticosteroid and a drug selected from the group consisting of an alkylating agent, a proteasome inhibitor, and combinations thereof.

11. The compound of formula (I) for use in a method of treatment according to any of claim 9 or 10,
wherein said alkylating agent is selected from the group consisting of nitrogen mustards, nitrosoureas and alkyl sulfonates, preferably is a nitrogen mustard, more preferably is cyclophosphamide;
wherein said corticosteroid is selected from the group consisting of dexamethasone, prednisolone and methylprednisolone, preferably is dexamethasone; and
wherein said proteasome inhibitor is selected from the group consisting of bortezomib, carfilzomib and ixazomib, preferably is bortezomib.

12. The compound of formula (I) for use in a method of treatment according to claim 11, wherein the compound of formula (I) is used in a combination treatment selected from the group consisting of:
the compound of formula (I)+bortezomib;
the compound of formula (I)+cyclophosphamide;
the compound of formula (I)+dexamethasone;
the compound of formula (I)+bortezomib+dexamethasone; and
the compound of formula (I)+cyclophosphamide+dexamethasone.

13. The compound of formula (I) for use in a method of treatment according to any of claims 1 to 12, wherein the compound of formula (I) is administered at days 1 to 7 of each week of a treatment cycle, preferably once or twice per day, and the other anticancer drug is administered at days 1 and 4 of each week of a treatment cycle, preferably once per day.

14. The compound of formula (I) for use in a method of treatment according to any of claims 1 to 12, wherein the compound of formula (I) is administered at days 1, 3, and 5 of each week of a treatment cycle, preferably once per day, and the other anticancer drug is administered at days 1 and 4 of each week of a treatment cycle, preferably once per day.

15. The compound of formula (I) for use in a method of treatment according to any of claims 1 to 12, wherein the compound of formula (I) is administered at days 1 and 4 of each week of a treatment cycle, preferably once per day, and the other anticancer drug is administered at days 1 and 4 of each week of a treatment cycle, preferably once per day.

16. The compound of formula (I) for use in a method of treatment according to any of claims 1 to 15, wherein the compound of formula (I) is formulated to be administered at a dosage from 0.1 mg/kg to 1 mg/kg, preferably from 0.25 mg/kg to 0.5 mg/kg; and
wherein, preferably, the compound of formula (I) is formulated for parenteral administration, preferably for intravenous, intramuscular, intraperitoneal, intrapreural or intravenous administration, more preferably for intravenous administration.

17. A pharmaceutical composition comprising the compound of formula (I) as defined in any of claims 1 to 6, and a pharmaceutically acceptable carrier or excipient, for use in a method of treatment according to any of claims 1 to 16.

18. A pharmaceutical composition comprising a compound of formula (I) as defined in any of claims 1 to 6, another drug, and a pharmaceutically acceptable carrier or excipient, for use in a method of treatment according to any of claims 8 to 16.


SEQUENCE LISTING (14 sequences)

SEQ ID NO: 1 - protein, 16 aa, Artificial Sequence
  Peptide
  MISC_FEATURE: It corresponds to the peptidic sequence (without the birradical ligand) of the compound S09
APKAVILKKAAAYILS

SEQ ID NO: 2 - protein, 16 aa, Artificial Sequence
  Peptide
  MISC_FEATURE: It corresponds to the peptidic sequence (without the birradical ligand) of the compound S14
APKAVIFKKAAAYILS

SEQ ID NO: 3 - protein, 16 aa, Artificial Sequence
  Compound S09
  MISC_FEATURE (4)..(4): The X residue at position 4 has formula (II) where R1 is -CH3 and it binds to the X residue at position 11 via a L birradical which is -(CH2)6-CH=CH-(CH2)3-
  MISC_FEATURE (11)..(11): The X residue at position 11 has formula (II) where R1 is -CH3
APKXVILKKAXAYILS

SEQ ID NO: 4 - protein, 16 aa, Artificial Sequence
  Compound S14
  MISC_FEATURE (4)..(4): The X residue at position 4 has formula (II) where R1 is -CH3 and it binds to the X residue at position 11 via a L birradical which is -(CH2)6-CH=CH-(CH2)3-
  MISC_FEATURE (11)..(11): The X residue at position 11 has formula (II) where R1 is -CH3
APKXVIFKKAXAYILS

SEQ ID NO: 5 - protein, 16 aa, Artificial Sequence
  Compound S19
  MISC_FEATURE (2)..(2): The X residue at position 2 has formula (II) where R1 is -CH3 and it binds to the X residue at position 9 via a L birradical which is -(CH2)6-CH=CH-(CH2)3-
  MISC_FEATURE (9)..(9): The X residue at position 9 has formula (II) where R1 is -CH3
AXKVVILKXATAYILS

SEQ ID NO: 6 - protein, 16 aa, Artificial Sequence
  Compound S21
  MISC_FEATURE (5)..(5): The X residue at position 5 has formula (II) where R1 is -CH3 and it binds to the X residue at position 12 via a L birradical which is -(CH2)6-CH=CH-(CH2)3-
  MISC_FEATURE (12)..(12): The X residue at position 12 has formula (II) where R1 is -CH3
APKVXILKKATXYILS

SEQ ID NO: 7 - protein, 16 aa, Artificial Sequence
  Compound S22
  MISC_FEATURE (6)..(6): The X residue at position 6 has formula (II) where R1 is -CH3 and it binds to the X residue at position 13 via a L birradical which is -(CH2)6-CH=CH-(CH2)3-
  MISC_FEATURE (13)..(13): The X residue at position 13 has formula (II) where R1 is -CH3
APKVVXLKKATAXILS

SEQ ID NO: 8 - protein, 17 aa, Artificial Sequence
  Compound S23
  MISC_FEATURE (9)..(9): The X residue at position 9 has formula (II) where R1 is -CH3 and it binds to the X residue at position 16 via a L birradical which is -(CH2)6-CH=CH-(CH2)3-
  MISC_FEATURE (16)..(16): The X residue at position 16 has formula (II) where R1 is -CH3
APKVVILKXATAYILXV

SEQ ID NO: 9 - protein, 16 aa, Artificial Sequence
  Compound S17
  MISC_FEATURE (7)..(7): The X residue at position 7 has formula (II) where R1 is -CH3 and it binds to the X residue at position 14 via a L birradical which is -(CH2)6-CH=CH-(CH2)3-
  MISC_FEATURE (14)..(14): The X residue at position 14 has formula (II) where R1 is -CH3
APKVVIXKKATAYXLS

SEQ ID NO: 10 - protein, 16 aa, Artificial Sequence
  Compound S18
  MISC_FEATURE (8)..(8): The X residue at position 8 has formula (II) where R1 is -CH3 and it binds to the X residue at position 15 via a L birradical which is -(CH2)6-CH=CH-(CH2)3-
  MISC_FEATURE (15)..(15): The X residue at position 15 has formula (II) where R1 is -CH3
APKVVILXKATAYIXS

SEQ ID NO: 11 - protein, 13 aa, Artificial Sequence
  Peptide
  MISC_FEATURE: wild type sequence
PKVVILKKATAYI

SEQ ID NO: 12 - protein, 16 aa, Artificial Sequence
  Peptide
  MISC_FEATURE: It corresponds to the peptidic sequence (without the birradical ligand) of the compounds IDP-P1708160 and IDP-P1708161
APKGVILKKAGAYILS

SEQ ID NO: 13 - protein, 16 aa, Artificial Sequence
  Compound IDP-P1708160
  MISC_FEATURE (4)..(4): The X residue at position 4 has formula (II) where R1 is -H and it binds to the X residue at position 11 via a L birradical which is -(CH2)-S-(CH2)8-S-(CH2)-
  MISC_FEATURE (11)..(11): The X residue at position 11 has formula (II) where R1 is -H
APKXVILKKAXAYILS

SEQ ID NO: 14 - protein, 16 aa, Artificial Sequence
  Compound IDP-P1708161
  MISC_FEATURE (4)..(4): The X residue at position 4 has formula (II) where R1 is -H and it binds to the X residue at position 11 via a L birradical which is -(CH2)-S-(CH2)-biphenyl-(CH2)-S-(CH2)-
  MISC_FEATURE (11)..(11): The X residue at position 11 has formula (II) where R1 is -H
APKXVILKKAXAYILS